(19) United States
(12) Patent Application Publication
(10) Pub. No.: US 2024/0307373 A1
(43) Pub. Date: Sep. 19, 2024

(54) METHODS OF USE AND COMPOSITIONS OF BISBENZYLISOQUINOLINES FOR THE TREATMENT OF MALIGNANCIES

(71) Applicant: Escend Pharmaceuticals, Inc., Menlo Park, CA (US)

(72) Inventors: Saira S. Bates, Napa, CA (US); Dennis Brown, Palo Alto, CA (US); Shawnya Michaels, El Granada, CA (US)

(21) Appl. No.: 18/605,750

(22) Filed: Mar. 14, 2024

Related U.S. Application Data
(60) Provisional application No. 63/453,063, filed on Mar. 17, 2023.
(60) Provisional application No. 63/453,068, filed on Mar. 18, 2023.

(51) Int. Cl. A61K 31/4748; A61K 31/506; A61K 45/06; A61P 35/02

(52) U.S. Cl. CPC A61K 31/4748; A61K 31/506; A61K 45/06; A61P 35/02

(57) ABSTRACT

The present disclosed invention provides pharmaceutical compositions and methods of use for bisbenzylisoquinolines such as 6,6′,7,12-tetramethoxy-2,2′-dimethyl-berbaman, and analogs, derivatives, isomers, and modified forms such as crystalline, salt forms, or a salt of this compound with a pharmaceutically acceptable acid or in combination with other agents to treat acute, chronic and pre-leukemic conditions as well as lymphomas and solid tumors. These include preneoplastic and neoplastic diseases and solid tumors including but not limited to acute myeloid leukemia (AML), acute lymphoblastic leukemia (ALL), chronic myeloid leukemia (CML), atypical chronic myeloid leukemia (aCML), and acute myeloid leukemia (AML), polycythemia vera (PV), chronic lymphoblastic leukemia (CLL), myeloproliferative syndrome (MPS), myelodysplastic syndrome (MDS), myeloproliferative neoplasms (MPN), myelofibrosis (MF), and polycythemia vera (PV).

### CROSS REFERENCE TO RELATED APPLICATIONS

This application is a U.S. Utility Patent application which claims the benefit of priority to both U.S. Provisional application No. 63/453,063, filed Mar. 17, 2023, entitled “Methods of Use and Compositions of Bisbenzylisoquinolines such as 6,6′,7,12-tetramethoxy-2,2′-dimethyl-berbaman or Analogs for the Treatment of Leukemic Stem Cells, Cancer Stem Cells and Precancerous Stem Cells” and U.S. Provisional application No. 63/453,068, filed Mar. 18, 2023, entitled “Leukemic Stem Cell Treatment,” each of which are hereby incorporated herein by reference in their entirety.

### BACKGROUND

While both systemic and local toxicities of cancer chemotherapeutics can limit their utility, treatment failure and cancer relapse are also the result of drug resistance and self-renewal properties of a small population of tumor cells identified as cancer stem cells (CSCs). Some properties of CSCs, such as cell surface markers (CD44, CD24, and CD133) have been identified that can allow the development of therapies better suited toward eradicating these cellular populations. CSCs have a survival reliance on certain signaling pathways (e.g., Wnt/β-catenin, Notch, NF-kB, NLRP3 and Hedgehog that control CSCs properties. Normal stem cells do not. For CSCs, this reduces apoptotic signals and increases proliferative ones, and they over-express drug-efflux pumps that allow escape from toxic chemotherapeutic concentrations. Moreover, the influence of the bone marrow microenvironment interaction with leukemic stem cells through the Wnt/β-catenin pathway, NF-kB as well as the NLRP3 inflammatory pathway provides the opportunity for therapeutic intervention. Knowing the survival characteristics of CSCs allows for the identification and development of therapeutics especially suited toward their elimination. Cancers for which CSCs-enriched populations have been identified and include brain, head and neck, breast, pancreatic, lung, liver, gastric, colorectal, lymphomas and leukemias.

Tetrandrine (6,6′,7,12-tetramethoxy-2,2′-dimethylberbam), also known as d-tetrandrine, TET, NSC #77037, and ES-3000 is a bis-benzylisoquinoline alkaloid, extracted from roots of the plant Stephania tetrandra S. moore. ES-3000 (Tetrandrine) is being developed by Escend Pharmaceuticals, Inc. (Escend) for the treatment of leukemias and related hematological malignancies including relapsed or refractory acute myeloid leukemia (AML), chronic myeloid leukemia (CML), myelodysplastic syndrome (MDS). and polycythemia vera (PV).

### FIELD OF INVENTION

Bis-benzylisoquinolines (BBI) represent a large class of alkaloids having two benzylisoquinoline units linked by ether bridges and include methylenoxy linkage or direct carbon-carbon bonding. BBIs have various biological activities. The present disclosed invention provides pharmaceutical compositions and methods of use for bisbenzylisoquinolines such as 6,6′,7,12-tetramethoxy-2,2′-dimethyl-berbaman (AKA tetrandrine, d-tetrandrine, TET, NSC #77037, and ES-3000), and analogs, derivatives. isomers, and modified forms such as crystalline, salt forms, or a salt of this compound with a pharmaceutically acceptable acid or in combination with other agents to treat acute, chronic and pre-leukemic conditions as well as lymphomas and solid tumors. These include preneoplastic and neoplastic diseases and solid tumors including but not limited to acute myeloid leukemia (AML), acute lymphoblastic leukemia (ALL), chronic myeloid leukemia (CML), atypical chronic myeloid leukemia (aCML), and acute myeloid leukemia (AML), polycythemia vera (PV), chronic lymphoblastic leukemia (CLL), myeloproliferative syndrome (MPS), myelodysplastic syndrome (MDS), myeloproliferative neoplasms (MPN), myelofibrosis (MF), and polycythemia vera (PV). The invention also relates to the use of bisbenzylisoquinolines such as 6,6′,7,12-tetramethoxy-2,2′-dimethyl-berbaman (tetrandrine), for the treatment and/or conditioning of patients for bone marrow transplant and hematopoietic stem cell transplantation (HSCT). These agents can be used as a single agent or in combination with other therapeutics such as alkylating agents, antimetabolites, anti-tumor antibiotics, topoisomerase inhibitors, mitotic inhibitors, and corticosteroids, or targeted therapeutics such as TKI and antibody directed chemotherapy for the treatment of cancer and leukemia where stem cells may be the cause of treatment resistance

### BACKGROUND OF THE DISCLOSED INVENTION

Indications such as MDS, MPN, CML, aCML, AML and PV are bone marrow diseases that affect blood-forming stem cells. The precursor of leukemic disease is MDS, which is not a single disease but a group of diseases that affect blood cell formation. In all forms of MDS, bone marrow aberration leads to low levels of blood cells circulating in the bloodstream. In a healthy person, the stem cells in the bone marrow make daughter cells. The daughter cells go through several stages of development within the marrow. Eventually, they mature into red blood cells (RBCs), white blood cells (WBCs), or platelets. These are released from the marrow to circulate in the bloodstream and perform important functions, such as carrying oxygen throughout the body, fighting infection, and protecting the body against bleeding by helping blood to clot.

In a person with MDS, the stem cells in the bone marrow do not function normally. Instead of producing healthy, mature RBCs, WBCs, and platelets, the marrow makes cells that tend to remain immature and die early. As a result, people with MDS have low levels of one or more types of blood cells in their bloodstream (cytopenia) such as anemia (low level of RBCs), leucopenia (low level of WBCs) and thrombocytopenia (low level of platelets). Low levels of blood cells, or low blood counts, cause the symptoms of MDS. The disease progresses over time in two ways. In most people with MDS, fewer and fewer healthy blood cells are produced or survive. This can lead to severe anemia (low RBCs), increased risk of infection (due to low WBCs), or risk of severe bleeding (due to low platelets). In about 30 percent of people with MDS (depending on the subtype), the number of very immature abnormal cells in the marrow (blast cells, or blasts) increases, and MDS transforms into acute leukemia. This risk of developing leukemia is referred to ‘low, moderate or high risk’ subtype of MDS.

The World Health Organization (WHO) system of MDS classification for subtypes of MDS is listed below:

Refractory cytopenia with unilineage dysplasia (RCUD). Too few RBCs (anemia) or too few WBCs (neutropenia) or too few platelets (thrombocytopenia) in blood.

Refractory anemia with ring sideroblasts (RARS). Ring sideroblasts are early stages of RBCs that have accumulated abnormal amounts of iron. WBC and platelet counts may be normal but have a low percentage of blasts in marrow and blood.

Refractory cytopenia with multilineage dysplasia (RCMD). Marrow shows dysplastic changes in the cells that make at least two types of blood cells: RBCs, WBCs, or platelets. Low percentage of blasts in marrow and blood. If multilineage dysplasia and ring sideroblasts, this is classified as RCMD-RS.

Refractory anemia with excess blasts-1 (RAEB-1) and refractory anemia with excess blasts-2 (RAEB-2). At least 5% (RAEB-1) or at least 10% (RAEB-2) but less than 20% blasts in marrow.

MDS associated with isolated abnormality of chromosome 5 [del(5q)]. Part of chromosome 5 is missing. Typically it means too few RBCs, low percentage of blasts in marrow and blood.

MDS, unclassifiable (MDS-U). This subtype means that marrow shows dysplastic changes in cells that make WBCs or platelets (but not in those that make RBCs). The patient has a normal percentage of blasts in the marrow and blood and the MDS does not fit into one of the other subtypes.

Some problems with blood-cell formation are not only myelodysplastic (having to do with abnormal production of cells in the marrow) or only myeloproliferative (having to do with over production of cells in the marrow). Instead, these problems have features of both MDS and MPN, they include the following:

Chronic myelomonocytic leukemia (CMML). The main feature of CMML is having too many myelocytes and monocytes, both types of WBCs, in blood.

Juvenile myelomonocytic leukemia (JMML). Similar to CMML, it occurs in young children. It causes high levels of myelocytes and monocytes.

Atypical chronic myeloid leukemia (aCML), BCR-ABL 1-negative. In aCML, the presence of too many granulocytes, a type of WBC, aCML is much like chronic myeloid leukemia (CML), except people with CML have a chromosome change called the Philadelphia chromosome that patients with aCML do not have.

MDS/MPN, unclassifiable. The disease has features of MDS and MPN and does not fit into one of the other subtypes. This subtype includes refractory anemia with ringed sideroblasts and thrombocytosis (RARS-T).

In CML, the BCR-ABL tyrosine kinase inhibitors (TKIs) imatinib mesylate (IM), nilotinib, and dasatinib, etc., have revolutionized the treatment of Philadelphia-positive (Ph+) leukemia in both CML and B-cell acute lymphoblastic leukemia (B-ALL) by targeting and disabling the proliferative signal coming from BCR-ABL. However, clinical resistance to these TKIs negates the potential curative results in Ph+ leukemia.

Resistance to TKIs is a problem for a subset of patients with CML Resistance is particularly important for the patients who develop the T315I BCR-ABL kinase domain (KD) mutation which represents approximately 15% of all mutations detected after failure to TKI. The T315I mutation results in resistance to imatinib mesylate (IM) and the second generation TKIs, including dasatinib (D), nilotinib (N), bosutinib (B), ponatinib and bafetinib.

In the case of AML, outcomes for adults with AML are poor, with a long-term overall survival (OS) of only 40-50% for younger patients and a median OS of less than one year for older patients. The addition of therapies to the standard remission induction regimen of cytarabine infusion with intermittent dosing of an anthracycline (7+3) has not resulted in significant added benefit. It has recently been recognized that leukemia stem cells (LSCs), which are capable of giving rise to identical daughter cells as well as differentiated cells, perpetuate and maintain AML. LSCs have different properties than the bulk AML population, making them difficult to eliminate with standard chemotherapy, and therefore a source of disease resistance and relapse.

### SUMMARY OF THE DISCLOSED INVENTION

Methods are disclosed for treating hematologic cancer patients comprising treating the patient with a bisbenzylisoquinoline before and/or after, in addition to treatment with standard of care therapies, including alkylating agents, antimetabolites, anti-tumor antibiotics, topoisomerase inhibitors, mitotic inhibitors and corticosteroids, or targeted therapeutics (e.g., TKIs) for the treatment of cancer and leukemia stem cells. Traditional chemotherapeutics include but are not limited to daunorubicin, doxorubicin, cytarabine, cisplatin, gemcitabine, vinblastine, etoposide, decitabine, azacytidine, venetoclax. Targeted therapeutics include but are not limited to tyrosine kinase inhibitors such as imatinib, dasatinib nilotinib, bosutinib and ponatinib. The bisbenzylisoquinoline treatment is preferably carried out until the patient demonstrates a hematological or cytological response to leukemia If the leukemic cells in a patient develop resistance to the TKI or other chemotherapeutics, bisbenzylisoquinoline therapeutic treatment is repeated, along with repeated treatment with TKIs or other chemotherapeutic regimens. The bisbenzylisoquinoline treatment reduces leukemic stem cell populations, inhibits the development of additional leukemic stem cells, and reduces or eliminate leukemic stem cells including those clonal populations that are resistant to TKI treatment or other chemotherapeutic regimens which would otherwise expand during treatments which did not include the bisbenzyliosquinoline. A clonal population containing the ber-abl genotype having the T315I mutation is an example of such a population. If necessary, the bisbenzylisoquinoline treatment is repeated until the patient demonstrates a hematological or cytological response to leukemia. Thereafter, the patient can be treated with the same or a different TKI or chemotherapeutic regimen for further improved patient outcomes with or without the bisbenzyliosquinoline treatment. These agents utilize different mechanisms of action and while response to treatments varies with these agents, serious adverse side-effect reactions can be present and limit treatment effectiveness.

The disclosed invention describes the use of bisbenzylisoquinolines such as 6,6′,7,12-tetramethoxy-2,2′-dimethyl-berbaman (tetrandrine) and derivatives, isomers and analogs, prodrugs modified forms such as salt forms, or a salt of this compound with a pharmaceutically acceptable acid in the inhibition of molecular signaling pathways involved in the survival, self-renewal and differentiation of cancer stem cells and leukemia stem cells including but not limited to the cellular targets or pathways Alox5, Stat3, Wnt/beta-catenin, Msr2, Blk, Myc, Survivn, Cyclin D, Osteopontin, Tenascin C, L1CAM, NF-kB, NLRP3, and CaMKII.

The disclosed invention describes the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid in the inhibition of molecular protein targets involved in the survival of cancer and leukemia stem cells including but not limited to lipoxygenases, Mcl-1, cyclin-D1, beta-catenin, Bcl-2, Bcl-xL NF-kB, CamKIIγ, pCaMKIIγ, NLRP3, and VEGF.

The disclosed invention describes the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid alone or in combination with chemotherapeutic or immunosuppressive agents including epoetin alpha+GCSF, azacitidine (Vildaza), decitibine (Dacogen), cedazuridine, Inqovi® (decitabine and cedazuridine), cytarabine, fludarabine, lenalidomide (Revlimid) for immunosuppressive, immunomodulatory and anti-inflammatory activities that enable treatment of MDS, MPN, CML, aCML, AML, PV, and related diseases.

The disclosed invention describes the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid alone or in combination with tyrosine kinase inhibitors including imatinib, dasatinib, nilotinib, bosutinib, ponatinib, AZD0530, NPB-001-05, AT9283, BAY43-9006, bafetinib, AP24534, lestaurtinib, tozasertib, danusertib, XL228, KW-2449, AT-9283, VE-465, DCC-2036, PKC412, PF-03814735 and vatalanib for treatment of solid tumor cancers, lymphomas, MPN, CML, aCML, AML, PV and related myelodysplastic diseases.

Tetrandrine is a preferred bisbenzylisoquinoline, although other bisbenzylisoquinoline analogs can be used. The initial treatment with tetrandrine is preferably about 2 to 15 mg/kg tetrandrine per day, more preferably 5 to 10 mg/kg tetrandrine per day. For oral delivery, the preferred initial treatment with tetrandrine could be expressed as a fixed dose of 360 mg per day, and more preferably, 540 mg per day. The tetrandrine treatment can be for 7 days or more. However, the treatment may be as long as 14-21 days consecutively in a 28 day cycle or chronically with daily administration lasting 6-8 weeks or up to 3 months. In some cases, the amount and/or duration may be less than 5 mg/kg or 350 mg tetrandrine per day and less than 7 days. The therapy can also be administered chronically. In addition, tetrandrine derivatives may be provided for parenteral use such as IV, as a bolus or infusion.

The foregoing methods can also be modified so that the treatment with TKI or chemotherapeutic regimen is supplemented with concurrent treatment with a bisbenzylisoquinoline. In such cases, (1) the amount of bisbenzylisoquinoline can be lower than that which would be used if administered alone, (2) the time for bisbenzylisoquinoline treatment can be reduced (e.g. 2-7 days for tetrandrine), or (3) the amount and time of bisbenzylisoquinoline treatment can be reduced. In addition, the amount of TKI or other chemotherapeutic regimens can also be lower than if administered alone or alternative dosing schedules may be employed.

The disclosed invention also includes methods to treat leukemic patients who have developed resistance to TKIs including imatinib, or chemotherapeutic regimens such as 7+3 (cytarabine for 7 days with daunorubicin for the first 3 days) or high-dose cytarabine for 5 days. The treatment with a bisbenzylisoquinoline is to reduce leukemic cell populations and inhibit the development or proliferation of leukemic stem cells that have acquired treatment resistance. Such patients may be contemporaneously treated with a TKI or other chemotherapeutic regimens, or subsequently treated with a TKI or other chemotherapeutic regimens after the patient demonstrates a hematological or cytological responses to leukemia. In addition, other anti-leukemia agents can be administered to the patient before, during, or after administration of bisbenzylisoquinoline or TKI or other chemotherapeutic regimens. Such additional treatment includes the use of inhibitors of SRC-kinases, and protein synthesis inhibitors such as omacetaxine, interferon-alpha, traditional and standard chemotherapeutics such as alkylating agents, antimetabolites, anti-tumor antibiotics, topoisomerase inhibitors, mitotic inhibitors and corticosteroids, or targeted therapeutics. Traditional chemotherapeutics include but are not limited to daunorubicin, doxorubicin, cytarabine, cisplatin, gemcitabine, vinblastine, etoposide, decitabine, azacytidine, venetoclax. Targeted therapeutics include but are not limited to tyrosine kinase inhibitors such as imatinib, dasatinib nilotinib, bosutinib, and ponatinib. Solid tumors and lymphomas which benefit from the treatment of bisbenzylisoquinolines such as 6,6′,7,12-tetramethoxy-2,2′-dimethyl-berbaman (tetrandrine) and derivatives, isomers and analogs, prodrugs modified forms such as salt forms, or a salt of this compound with a pharmaceutically acceptable acid alone or in combination with chemotherapeutics or targeted therapies include triple negative breast cancer (TNBC), other breast cancers, pancreatic cancer, brain cancers, head and neck cancer, lung cancers, liver cancer, gastric cancer and colorectal cancers, all of which have cancer stem cell enriched populations with known surface antigens for identification as biomarkers.

Further, the disclosed invention describes novel improvements, pharmaceutical ingredients, dosage forms, excipients, solvents, diluents, drug delivery systems, preservatives, toxicity monitoring and ameliorization, and techniques or agents to circumvent or reduce toxicity. The disclosed invention also relates to the use of drug delivery systems, prodrugs, polymer conjugates, routes of administration, antibody conjugates, and other agents to potentiate the activity of the compounds or inhibit the repair of suboptimal cellular effects or sublethal damage or to “push” the cell into more destructive cellular phases such as apoptosis. In some cases, the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid in conjunction with radiation or other conventional chemotherapeutic agents or biotherapeutic agents including but not limited to antibodies, vaccines, cytokines, lymphokines, gene and antisense therapies, CAR-TCR cell therapies, etc., would provide novel approaches and significant improvement for suboptimal therapeutics.

Bisbenzylisoquinolines such as 6,6′,7,12-tetramethoxy-2,2′-dimethyl-berbaman (tetrandrine) and derivatives, isomers and analogs, prodrugs modified forms such as salt forms, or a salt of this compound with a pharmaceutically acceptable acid may be prepared either synthetically, semi-synthetically, biochemically or through extraction processes from plant material containing the agent, precursors or intermediates, including but not limited to, Stephania tetrandra and Stephania abuta.

Pharmaceutically acceptable salts may include salts with inorganic acids, such as hydrochloride, sulphate, nitrate and phosphate, or organic acids, such as acetate, propionate, succinate, oxalate, benzoate, fumarate, maleate, methane sulphonate, isethionate, theophyllineacetate, salicylate, phenolphthalinate and methylene-bis-b-hydroxynaphthoate, and the like or substitution derivatives of these derivatives. These may also include citrate and malate salt forms.

The disclosed invention also relates to pharmaceutical compositions consisting of bisbenzylisoquinolines such as 6,6′,7,12-tetramethoxy-2,2′-dimethyl-berbaman (tetrandrine) and derivatives, isomers and analogs, prodrugs modified forms such as crystalline, salt forms, or a salt of this compound with a pharmaceutically acceptable acid, in the pure state or in the form of a composition in which it is combined with any other pharmaceutically compatible product, which can be inert or physiologically active. The medicaments according to the disclosed invention may be employed orally, parenterally, rectally, topically or as a patch.

As solid compositions for oral administration, tablets, pills, powders (solutions, gelatin capsules, wafer capsules), medical foods or granules may be used. In these compositions, the active principle according to the disclosed invention is mixed with one or more inert excipients or diluents such as starch, cellulose, sucrose, lactose, or silica, with or without under a stream of argon. These compositions may also comprise substances other than diluents, for example, one or more lubricants such as magnesium stearate or talc, a coloring, a coating, or a varnish.

As liquid compositions for oral administration, pharmaceutically acceptable solutions, suspensions, emulsions, syrups, or elixirs, containing inert diluents such as water, ethanol, glycerol, vegetable oils, or liquid paraffin, etc., may be used. These compositions can comprise substances other than diluents, for example, wetting agents, sweeteners, thickeners, flavors, or stabilizing products.

The sterile compositions for parenteral administration can preferably be solutions, aqueous or non-aqueous, suspensions or emulsions. As a solvent or vehicle, water, propylene glycol, a polyethylene glycol, vegetable oils, olive oil, injectable organic esters, for example, ethyl oleate, or other suitable organic solvents may be employed such as DMSO, DMA, and ethanol. These compositions can also contain adjuvants, especially wetting, isotonising, emulsifying, dispersing, and stabilizing agents. The sterilization may be carried out in several ways, for example by aseptic filtration, by incorporating sterilizing agents in the composition, by irradiation or by heating. They may also be prepared in the form of sterile solid compositions which can be dissolved at the time of use in co-solvents, sterile water or any other sterile injectable medium.

The compositions for rectal administration are suppositories or rectal capsules that contain, apart from the active product, excipients such as cocoa butter, semi-synthetic glycerides or polyethylene glycols.

The compositions for topical administration can be, for example, creams, lotions, mouthwashes, nasal drops, aerosols or patches.

The doses depend on the effect sought, on the duration of the treatment, and the administration route used or if used as a single agent or in combination with other therapies. The doses are generally between 5 and 1000 mg per day administered orally for an adult, with single doses ranging from 20 to 450 mg of active substance each time, which could be 3 times per day. Generally speaking, the doctor and/or regulatory health authorities will determine the appropriate dosage, in accordance with the age and weight and all other factors distinctive to the subject to be treated including the disease state of the patient or approved regulatory guidelines.

Tetrandrine (6,6′,7,12-tetramethoxy-2,2′-dimethylberbam) or ES-3000 is being developed by Escend Pharmaceuticals, Inc. (Escend). It may be useful for the treatment of relapsed or refractory AML, MDS, and PV in combination with ASTX727, which is an oral fixed-dose combination of Decitabine (DEC) and Cedazuridine, recently FDA approved, for use in Myelodysplasia. The combination of two oral agents, with an established safety profile and potential combinatorial efficacy, would be a novel and much-needed approach to the treatment of low, intermediate and high-risk MDS or AML.

ASTX727 is the tablet referred to and is the investigational drug name for Iqovi [Decemberitabine (35 mg) and cedazuridine (100 mg)] tablet. The combination of ES-3000 at varying dosages can be administered with ASTX727. The ES-3000 schedule for administration can be once, twice or three times per day and can vary from about 1-14 days to about 1-7 days in combination with administration of the standard does for ASTX727 (Inqovi®) for about 1-5 days, about 1-4 days, about 1-3 days and about 1-2 days. The dosage of ES-3000 can be between 20-100 mg, 25-90 mg, 30-80 mg, 40-60 mg, as well as doses from about 20 mg, 30 mg, 40 mg, 50 mg, 60 mg, 70 mg, 80 mg, 90 mg, and 100 mg as well as 105 mg, 110 mg, 115 mg, 120 mg at once, twice or three times/day. It can also be administered chronically for weeks to months.

### BRIEF DESCRIPTION OF THE DRAWINGS

There are several known mechanisms of action of Tetrandrine including inhibition of calcium channels and activated big potassium channels, which are commonly overexpressed in malignancies and have been demonstrated to play a role in cancer and leukemia (Arcangeli 2010, Arcangeli 2012, Wang 1995, Huber 2013 Lang 2014, Döhner 2015). Tetrandrine has also been reported to reverse of P-glycoprotein (P-gp) mediated drug resistance (Zhu 2005, Jin 2005) and though Tetrandrine itself is not a substrate of P-gp, it directly inhibits the function of Pgp in a dose-dependent manner, without altering the protein expression level of P-gp (Susa 2010). More recently, Tetrandrine has been demonstrated to inhibit the Wnt/β-catenin pathway (Simon 2005, He 2011, Xu 2012), which has been identified as a new target for AML. Wnt/β-catenin is essential for the survival and self-renewal of leukemic stem cells in AML (Griffiths 2010, Wang 2010). In addition, Tetrandrine has been demonstrated to bind competitively to calmodulin (CaM) and may be a novel CaM antagonist (Ma 2013). Calmodulin protein-dependent kinase (CaMKIIγ) is overexpressed in leukemic stem cells and blast cells, and regulates the Wnt/β-catenin and STAT3 pathways (Si 2008).

Anticancer activity has been demonstrated in vitro with TET against human breast, colon carcinoma, and hepatoma cell lines with an IC50 range from 1.5-10.4 μM and inhibits drug efflux and increases intracellular drug accumulation in MDR overexpressed cancer cells. TET has also been shown to inhibit wnt/β-catenin signaling and tumor growth in human colorectal (HCT116) cells. In the same study, TET exhibited synergistic anticancer activity with 5-FU and reduced migration and invasion capabilities of HCT116 cells. (He 2011). In the NCI 60 cell line screen, a 50% growth inhibition (Log GI50) ranged from −5.0 to −6.6 (0.25-10 μM was demonstrated in leukemia cell lines (Developmental Therapeutics Program, NIH/NCI). TET has been reported to have a potent and specific activity in the reversal of P-glycoprotein-mediated drug resistance. In colorectal mouse xenografts, TET reduced tumor growth by inducing apoptosis (Wu 2010).

Published clinical data support the safety and efficacy of Tetrandrine in oncology patients and provide another compelling rationale for clinically assessing Tetrandrine in leukemia. A phase I dose-escalation study was conducted by the National Cancer Institute (NCI) under IND 9613 (also known at 089613). Thirty-two patients with advanced solid tumors were administered IV doses of Tetrandrine ranging from 50 to 875 mg/m2. The recommended dose for a Phase II study was 300 mg/m2 IV, a single dose repeated weekly; or 200 mg/m2 IV, daily ×5, repeated every 2 weeks (IND 9613 Tetrandrine Investigators Reports). Further supporting published reports include clinical studies using TET under the trademarked name CBT-1® in the US (Oldham 1998, Oldham 2000, Kelly 2012) and two studies in China (Xu 2006, Liu 2012). The drug known as CBT-1 is the same drug substance (NSC 77037) used in US Phase I study (IND #089613). CBT-1 had been publicly disclosed as NSC-77037 (tetrandrine) by (Fanelli 2016)

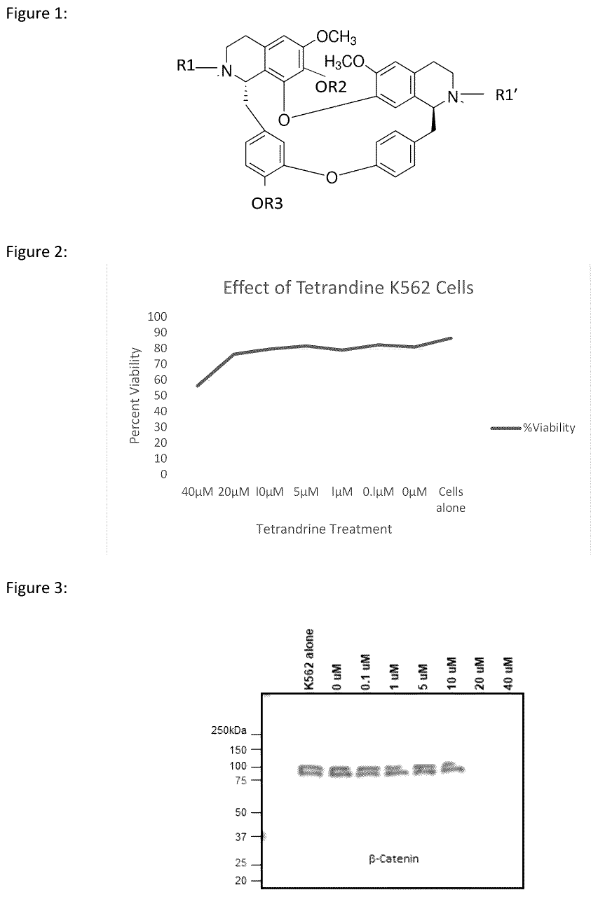

Chemical Structure of tetrandrine is illustrated in FIG. 1. Tetrandrine is the generic name for NSC 77037, d-tetrandrine with the chemical structure written as, 6′,7,12-tetramethoxy-2,2′-dimethyl-berbaman. The IUPAC name is 9,20,21,25-tetramethoxy-15,30-dimethyl-7,23-dioxa-15,30-diazaheptacyclo[22.6.2.23,6.18,12.114,18.027,31.022,33]hexatriaconta-3,5,8(34),9,11,18,20,22(33),24(32), 25,27(31),35-dodecaene, C38H42N2O6 is the chemical formula with a molecular weight of 622.76 and has CAS Registry No. 518-34-3.

The disclosed invention is based, in part, upon the discovery that the bisbenzylisoquinoline, tetrandrine, reduces leukemic stem cell populations, including those that have developed resistance to TKI treatment or other chemotherapeutic regimens. This discovery provides for anti-stem cell treatment protocols wherein a bisbenzylisoquinoline is administered to a leukemia patient to reduce leukemic stem cell populations followed by treatment with a TKI or other chemotherapeutic regimen. If resistance to the TKI or other chemotherapeutic regimen develops, treatment with the same or a different bisbenzylisoquinoline reduces the leukemic stem cell population, including TKI resistant leukemic stem cells, and leukemic stem cells resistant to other chemotherapeutic regimens. After that, treatment with the same or a different TKI, or other chemotherapeutic regimens, can be resumed. This cycle can be repeated as necessary to improve the hematological and cytogenetic responses.

The disclosed invention is also based, in part, on the discovery that leukemic patients who have developed resistance to TKIs or other chemotherapeutic regimens can be treated with a bisbenzylisoquinoline to reduce leukemic cell population and inhibit the development of leukemic stem cells that have acquired such resistance. Such patients may be contemporaneously treated with a TKI or other chemotherapeutic regimens, or subsequently treated with a TKI or other chemotherapeutic regimens after the patient demonstrates a hematological or cytological response to leukemia. Repeated cycles of such therapies may be employed to induce durable progression-free responses.

Other anti-leukemia agents can be administered to the patient before, during, or after the administration of bisbenzylisoquinoline or TKI or other chemotherapeutic regimens. Such additional treatment includes the use of inhibitors of SRC-kinases, aurora kinases, immunomodulators such as interferon-alpha, conventional chemotherapeutics such as hydroxyurea, cytarabine (ara-C), daunorubicin, doxorubicin, hypomethylating agents, decitabine, 5-azacytidine, venetoclax, liposomal formulations of anthracyclines with fraudulent nucleosides such as CPX-351(Vyxeos®) and the like.

Bisbenzylisoquinolines such as tetrandrine can be used in myeloablative conditioning to reduce toxicity and improve outcomes of hematopoietic stem cell transplantation (HSCT) procedures, with curative intent for patients with leukemia. Use of bisbenzylisoquinolines in myeloablative conditioning prior to HSCT can be before, after and in addition to myeloablative agents and procedures including but not limited to cyclophosphamide, busulfan, fludarabine, melphalan, clofarabine, amsacrine, cytarabine, decitabine, cedazuridine and radiation.

The disclosed invention describes the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid in the inhibition of molecular signaling pathways involved in the survival, self-renewal and differentiation of cancer stem cells and leukemia stem cells including but not limited to Alox5, Stat3, Wnt/beta-catenin, Msr2, Blk, Myc, Survivn, Cyclin D, Osteopontin, Tenascin C, L1CAM and CaMKII.

The disclosed invention describes the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid in the inhibition of molecular protein targets involved in the survival of cancer and leukemia stem cells including but not limited to lipoxygenases, Mcl-1, cyclin-D1, beta-catenin, Bcl-2, Bcl-xL NF-kappaB, CamKIIγ, pCaMKIIγ and VEGF.

The disclosed invention describes the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid alone or in combination with immunosuppressive agents including epoetin alpha+GCSF, Azacitidine (Vildaza), Decitibine (Dacogen), Lenalidomide for immunosuppressive and anti-inflammatory activities that enable treatment of MDS, MPN, CML, aCML, AML, PV and related diseases. These agents utilize different mechanisms of action and while the clinical response to treatments varies with these agents, serious adverse side-effect reactions can and may limit treatment effectiveness.

The disclosed invention describes the use of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid alone or in combination with tyrosine kinase inhibitors including imatinib, dasatinib, nilotinib, bosutinib, ponatinib, AZD0530, NPB-001-05, AT9283, BAY43-9006, bafetinib, AP24534, lestaurtinib, tozasertib, danusertib, XL228, KW-2449, AT-9283, VE-465, DCC-2036, PKC412, PF-03814735 and vatalanib for treatment of solid tumor cancers, MPN, CML, aCML, AML, PV and related diseases. These agents utilize different mechanisms of action and while response to treatments vary with these agents, serious adverse side-effect reactions are present and limit treatment effectiveness.

Solid tumors that benefit from the treatment of bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid alone or in combination with chemotherapeutics or targeted therapies include TNBC, other breast cancers, Hodgkins Lymphomas, Non-Hodgkins Lymphomas, multiple myeloma, pancreatic cancer, brain cancers, head and neck cancer, lung cancers, liver cancer, gastric cancer and colorectal cancers, all of which have cancer stem cell-enriched populations with known surface antigens for identification.

As used herein, leukemia refers to CML, AML, ALL, CLL, and APL. Leukemia also includes pre-leukemic syndromes such as MDS, MPN, and PV.

As used herein, TKI refers to any thymidine kinase inhibitor. Examples of TKIs include, but are not limited to, imatinib mesylate (IM) and second-generation TKIs, including dasatinib (D), nilotinib (N), bosutinib (B) and befatinib.

As used herein, other chemotherapeutic regimens include, but are not limited to, treatment with the 7+3 regimen (7 days of cytarabine with the first 3 days including daunorubicin), liposomal formulations of cytarabine with daunorubicin, high-dose cytarabine given for 5 days, hypomethylating agents such as 5 azacytidine, cedazuridine and decitabine.

As used herein, a leukemic stem cell refers to a pluripotent stem cell characterized by genetic transformation resulting in unregulated cell division. For example, in CML, the BCR-ABL fusion gene (Philadelphia chromosome). Leukemic stem cells may be defined by surface antigen profiles which include CD34+CD38-CD47+CCL-1+CD96+TIM3+CD32+CD25+.

As used herein, a cytological response to treatment with bisbenzylisoquinolines and/or TKI or other chemotherapeutic regimens is a response that occurs in the bone marrow rather than just in the peripheral blood. There are at least three cytological responses: (1) a cytogenetic response (CR); (2) a major cytogenetic response (MCR); and a complete cytogenetic response (CcyR). Determination of such responses is based on the measurement of the number of peripheral blood and/or bone marrow cells having a marker that is associated with a particular leukemia. Such markers include cell surface antigens, aberrant proteins, and genetic modifications. A cytogenetic response occurs if the number or percentage of cells with such a marker decreases during treatment. In the case of CML, a major cytogenetic response occurs if the number or percentage of such cells falls below 35%. A complete cytogenetic response occurs when no cells containing the marker are detected. For example, CML is characterized by the Ph+ chromosome. A cytogenetic response has occurred if the number of Ph+ chromosomes decreases at all during treatment. If the Ph+ percentage drops to 35% or less, it is considered a major cytogenetic response; 0% Ph+ is a complete cytogenetic response.

As used herein, a hematological response occurs when there is a change in the white blood cell count of a patient following treatment with bisbenzylisoquinolines and/or TKI or other chemotherapeutic regimens. In the case of CML, the change in white blood cell count can be in the peripheral blood and the bone marrow although the change may be observed only in the peripheral blood. The response can be a partial reduction in white cell count or a complete reduction to normal values (e.g. 10,000-12,000 cells per ml.)

As used herein, a molecular response, in the case of CML, occurs when there is a reduction to the BCR-ABLI transcript levels following treatment with bisbenzylisoquinolines and/or TKI. Following quantitative real-time PCR (qRT-PCR) analysis, the BCR-ABL1/control gene transcript ratio is determined using the International Scale (IS) standardized baseline. A major molecular response (MMR) occurs when there is a greater or equal reduction of 3log10 (≤0.10% IS) in BCR-ABL1 transcripts. Currently, there is no universal definition of complete molecular response (CMR).

As used herein, minimal residual disease (MRD) is the name given to small numbers of leukemic cells (cancer cells from the bone marrow) that remain in the patient during treatment, or after treatment when the patient is in remission (no symptoms or signs of disease). It is the major cause of relapse in cancer and leukemia. MRD is known to be enriched in leukemic stem cells. In CML, the presence of MRD is determined through the detection of the t(9;22) of which is considered standard of care for all patients with CML.

As used herein, the term bisbenzylisoquinolines includes all members of that chemical family including alkaloid derivatives of the Menispermaceae family, Stephania tetrandra S. moore, and other related species.

The medicaments according to the invention consist of bisbenzyliosquinolines such as 6,6′,7,12-tetramethoxy-2,2′-dimethyl-berbaman (tetrandrine) and derivatives, isomers, prodrugs and analogs, modified forms such as salt forms, or a salt of this compound with a pharmaceutically acceptable acid, in the pure state or in the form of a composition in which it is combined with any other pharmaceutically compatible product, which can be inert or physiologically active. The medicaments according to the invention may be employed orally, parenterally, rectally, topically or as a patch.

The bisbenzylisoquinoline formulations include those suitable for oral, topical or parenteral (including subcutaneous, intramuscular, and intravenous) administration. The formulations may conveniently be presented in unit dosage form and may be prepared by conventional pharmaceutical techniques. Such techniques include the step of bringing the active ingredient into association with the pharmaceutical carrier(s) or excipient(s). In general, the formulations are prepared by uniformly and intimately bringing into association the active ingredient with liquid carriers or finely divided solid carriers, emulsions, or both, and then, if necessary, shaping the product.

Bisbenzylisoquinoline formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc. They may be formulated for immediate release, sustained release, or controlled retention.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated to provide a slow or controlled release of the active ingredient therein for minutes to hours to days.

When oral formulations are used, the oral bisbenzylisoquinoline dosage form preferably is administered to a host in the range of 0.05-15.0 mg/Kg. In a preferred embodiment, the bisbenzylisoquinoline is administered to a host in the range of 2 to 10.0 mg/Kg. In a further preferred embodiment, the bisbenzylisoquinoline is administered to a host in the range of 6-10 mg/Kg, or as 420 mg-700 mg, and is administered once or split into increments to be given multiple times per day.

Bisbenzylisoquinoline formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules, and tablets of the kind previously described. The unit parenteral dose may contain 280-1,050 mg of bisbenzylisoquinoline, more preferred 420-700 mg per unit dose.

Bisbenzylisoquinoline preferred unit dosage formulations are those containing a daily dose or unit, daily sub-dose or multidose use, as herein above recited, or an appropriate fraction thereof, of the administered ingredient.

It should be understood that in addition to the ingredients, particularly mentioned above, the formulations may include other agents conventional in the art having regard to the type of formulation in question, for example, those suitable for oral administration may include flavoring agents, slow-release, and controlled release excipients, and coating and formulation technologies.

Further, the disclosed invention describes novel improvements, pharmaceutical ingredients, dosage forms, excipients, solvents, diluents, drug delivery systems, preservatives, toxicity monitoring and ameliorization, and techniques or agents to circumvent or reduce toxicity. The disclosed invention also relates to the use of drug delivery systems, novel prodrugs, polymer conjugates, novel routes of administration, antibody conjugates and other agents to potentiate the activity of the compounds or inhibit the repair of suboptimal cellular effects or sublethal damage or to “push” the cell into more destructive cellular responses such as apoptosis. In some cases, the use of bisbenzylisoquinolines such as 6,6′,7,12-tetramethoxy-2,2′-dimethyl-berbaman (tetrandrine), and derivatives, isomers, and analogs, modified forms such as salt forms, or a salt of this compound with a pharmaceutically acceptable acid in conjunction with radiation or other conventional chemotherapeutic agents or biotherapeutic agents including but not limited to antibodies, vaccines, cytokines, lymphokines, gene and antisense therapies, CAR-T, etc., would provide novel approaches and significant improvement for suboptimal therapeutics.

Bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid may be prepared either synthetically, semi-synthetically, biochemically or through extraction processes from plant material containing the agent, including but not limited to, Stephania tetrandra and Stephania abuta.

Pharmaceutically acceptable salts may include salts with inorganic acids, such as hydrochloride, sulphate, nitrate and phosphate, or organic acids, such as acetate, propionate, succinate, oxalate, benzoate, fumarate, maleate, methane sulphonate, isethionate, theophylline sodium acetate, salicylate, phenolphthalinate and methylene-bis-b-hydroxynaphthoate, or substitution derivatives of these derivatives. These may also include citrate and malate salt forms.

The disclosed invention also relates to pharmaceutical compositions consisting of bisbenzylisoquinolines such as 6,6′,7,12-tetramethoxy-2,2′-dimethyl-berbaman (tetrandrine) and derivatives, isomers and analogs, prodrugs modified forms such as crystalline, salt forms, or a salt of this compound with a pharmaceutically acceptable acid, in the pure state or in the form of a composition in which it is combined with any other pharmaceutically compatible product, which can be inert or physiologically active. The medicaments according to the disclosed invention may be employed orally, parenterally, rectally, topically or as a patch.

As solid compositions for oral administration, tablets, pills, powders (gelatin capsules, wafer capsules), medical foods or granules may be used. In these compositions, the active principle according to the disclosed invention is mixed with one or more inert diluents or excipients such as starch, cellulose, sucrose, lactose or silica, with or without under a stream of argon, or nitrogen. These compositions may also comprise substances other than diluents or excipients. For example, one or more lubricants such as magnesium stearate or talc, a coloring, a coating or a varnish may be used.

As liquid compositions for oral administration, pharmaceutically acceptable solutions, suspensions, emulsions, syrups or elixirs, containing inert diluents such as water, ethanol, glycerol, vegetable oils or liquid paraffin, may be used. These compositions can comprise substances other than diluents, for example, wetting, sweetening, thickening, flavoring agents or stabilizing additives may be employed.

The sterile compositions for parenteral administration can preferably be solutions, aqueous or non-aqueous, suspensions or emulsions. As a solvent or vehicle, water, propylene glycol, a polyethylene glycol, vegetable oils, especially olive oil, injectable organic esters, for example ethyl oleate, or other suitable organic solvents may be employed. These compositions can also contain adjuvants, especially wetting, isotonising, emulsifying, dispersing and stabilizing agents.

The sterilization may be carried out in several ways, for example by aseptic filtration, by incorporating sterilizing agents in the composition, by irradiation or by heating. They may also be prepared in the form of sterile solid compositions which can be dissolved at the time of use in co-solvents, sterile water or any other sterile injectable medium.

The compositions for rectal administration are suppositories or rectal capsules that contain, apart from the active product, excipients such as cocoa butter, semi-synthetic glycerides or polyethylene glycols.

The compositions for topical administration can be, for example, creams, lotions, mouthwashes, nasal drops, aerosols, or patches.

The doses depend on the effect sought on the duration of the treatment, and the administration route used. They are generally between 5 and 1000 mg per day administered orally for an adult, with single doses or multiple doses per day ranging from 5 to 450 mg of active substance at each dosing. The doctor will determine the appropriate dosage per age and weight and all other factors distinctive to the subject to be treated including the indication, and toxicology concerns as well as guidance from regulatory agents such as the FDA and EMA.

### EXAMPLES

Tetrandrine has an inhibitory activity against CML stem cells and is highly effective in treating CML induced by BCR-ABL in mice. FIG. 1 illustrates the structure of Tetrandrine.

### Example 1

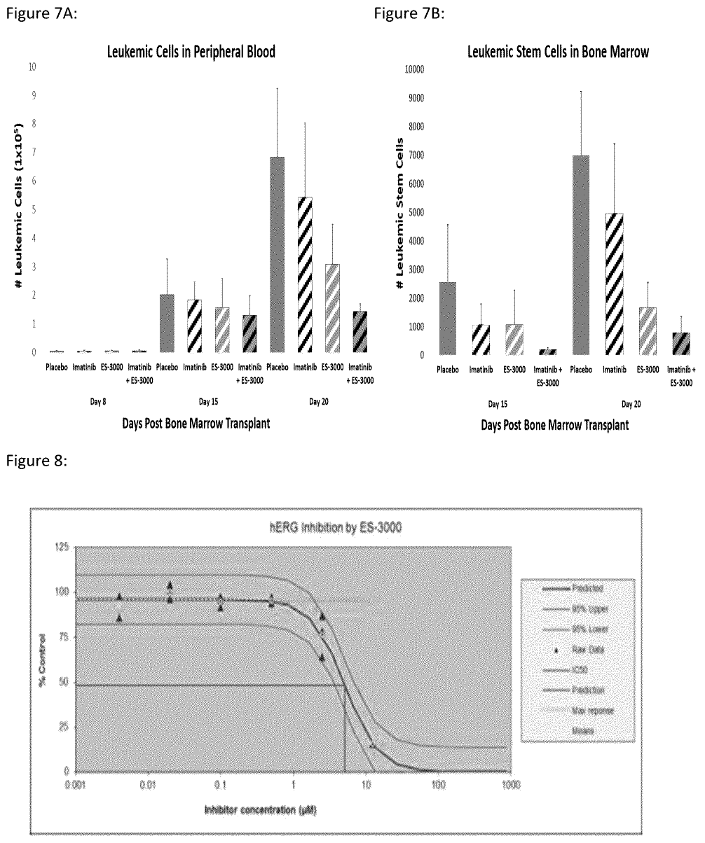

This example demonstrates that tetrandrine reduces the development of circulating leukemic cells, reduces spleen weight, and reduces the development of LSCs in a murine model of CML. The method is as follows: 20 C57BL/6 mice were used as donors for bone marrow transduction. They were primed by intravenous injection with 5-fluorouracil four days before collecting cells. Bone marrow cells were harvested from femurs and tibias. Bone marrow cells were infected with a retrovirus containing MSCV-BCR-ABL-IRES-GFP twice 60 recipient mice were lethally irradiated by two doses of 550cGy before bone marrow transplantation by intravenous injection with 500,000 cells/mouse. Mice were treated one week after bone marrow transplantation 15 mice in each group
Placebo: 3 times per day, 2 times with water and 1 time with 0.1N HCl, 6 hours apart each time
Imatinib: 100 mg/kg in water, 2 times per day, 12 hours apart
Tetrandrine: 150 mg/kg in 0.1N HCl, 1 time per day
Combo of Imatinib+ES3000: 3 times per day—2 times with Imatinib and 1 time with Tetrandrine, 6 hours apart. FIGS. 7A and 7B illustrate number of leukemic cells in the peripheral blood and leukemic stem cells, respectively vs., days following bone marrow transplant in C57BL/6 mice.

### Example 2

This example demonstrates that tetrandrine reduces the expression of β-catenin in K562 (human myeloid leukemia) cells. Levels of β-catenin and β-actin proteins in untreated and tetrandrine-treated K562 cell lines by Western blot.

Antibodies for Western Blot analysis against β-catenin and β-actin were purchased from Santa Cruz Biotechnology (Santa Cruz, CA) Protein lysates were prepared by lysing cells in RIPA buffer and immunoprecipitation.

FIG. 3 illustrates the results of Example 2.

### Example 3

This example demonstrates that tetrandrine eliminates BCR-ABL1+ secondary colonies in a Colony Forming Cell (CFC) assay of human CML bone marrow cells.

Clonogenic progenitors were set up using buman CML bone marrow-derived cells in the methylcellulose-based media formulation described above. Test articles were added to the medium to give final concentrations as described above. Standard cultures containing IMDM+10% FBS as well as solvent control cultures containing DMSO were also initiated. Following addition of the test compounds and cells, the tubes were vortexed to ensure equal distribution of compound and cells throughout the matrix. The cultures were set up in triplicate at 0.1×10⁶ cells per culture (lot #BBM1000-E1110025705111110G4) for each control and each compound concentration. The cultures were placed in a humidified incubator (37° C., 5% CO₂). Following 14-16 days in culture, the colonies were assessed and scored by trained personnel.

a. Replating:

Following enumeration of the erythroid and myeloid progenitors, individual CFU-GM colonies were plucked from the matrix and diluted in 20 μL of IMDM+10% FBS. The colony was dispersed in this medium by gentle pipetting. This cellular mixture was then added to a well of a 96 well plate containing 180 μL of the ColonyGel medium described above. Colonies were plucked from the solvent control cultures as well as 24 wells form the original cultures treated with 10 μM tetrandrine (the concentrations closest to the IC₅₀ value for both) The 96 well plates were placed into a humidified incubator for 14 days after which time, each well was evaluated for secondary colony growth. Both the number of wells which supported clonal growth as well as colony number per well were recorded.

### Colony Harvest, RNA Isolation and cDNA Conversion

After 14-16 days in culture, individual colonies were plucked from the solvent control and tetrandrine at 10 μM test condition into 75 μL of buffer RLT (Qiagen). Secondary replated colonies were collected and washed with 2 mL of 1× PBS, frozen at −80° C. and then lysed with 75 μL of buffer RLT (Qiagen). RNA was isolated using the Rneasy Micro kit according to the manufacture's instructions. Because the cell numbers in each colony were low, 20 ng of carrier RNA was included in each reaction to promote a better RNA yield. After elution, the entire isolation was subjected to a cDNA conversion using the high-capacity cDNA conversion kit (Life Technologies).

### Analysis for bre-abl Transcripts Using qPCR

Using cDNA derived from both primary and secondary replated colonies, qPCR assay was used to determine whether colonies were positive for ber-abl transcripts. Primers and probes were designed based on the publication from Luthra, et.al. and are designed to anneal to ber-abl (target) and abl (endogenous control) cDNA. Each target was assayed in duplicated in a 20 μL reaction volume. Samples were denatured at 95° C. for 10 min and then amplified for 40 rounds of 95° C. for 15 s and 60° C. for 60 s. FIG. 5 illustrates the results of Example 3

### Example 4

This example demonstrates that tetrandrine will improve survival in mice with BCR-ABL-WT-induced CML

The results of the experiments as set forth in Examples 1-4 demonstrate that: (a) tetrandrine reduces the development of circulating leukemic cells (b) reduces spleen weights (c) reduces the development of leukemic stem cells in a murine model of CML; (d) tetrandrine reduces the expression of β-catenin in K562 cells; (e) tetrandrine eliminates BCR-ABL1+ secondary colonies in a Colony Forming Cell (CFC) assay of human CML bone marrow cells.

### BRIEF DESCRIPTION OF THE DRAWINGS

FIG. 1 illustrates the chemical structure of ES-3000, also known as Tetrandrine (TET).

FIG. 2 graphically illustrates the effect of TET treatment on K562 cells as percent cell viability.

FIG. 3 illustrates a PAGE showing β-catenin protein exists as a doublet in all K562 cell extracts, including untreated cells.

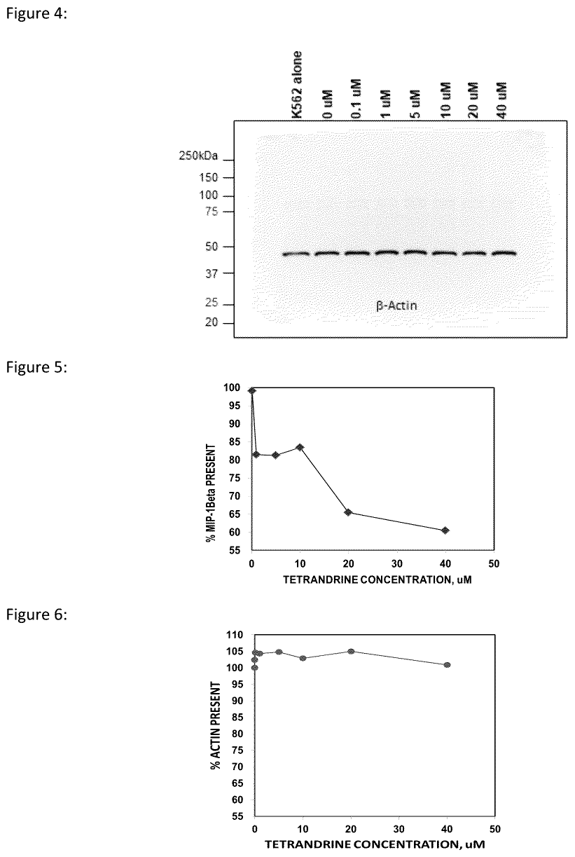

FIG. 4 illustrates a PAGE showing a clear beta-Actin band evident in all extracts of K562 cells treated with 0 to 40 uM tetrandrine.

FIG. 5 presents a plot depicting beta-catenin level was significantly less in extracts of K562 cells treated with >10 uM tetrandrine.

FIG. 6 presents a plot depicting beta-Actin level in extracts of K562 cells treated with up to 40 uM tetrandrine was not affected significantly by drug treatment

FIG. 7A illustrates a bar graph of samples of circulating leukemic blood cells from recipient mice following treatment with Tetrandrine or Imatinib+Tetrandrine.

FIG. 7B illustrates a bar graph of samples of leukemic stem cells from recipient mice after treatment with Tetrandrine or Imatinib+Tetrandrine on day 8 after bone marrow transplantation.

FIG. 8 graphically illustrates electrophysiology measurements of CHO cells stably transfected with hERG to examine the potential of a test article to inhibit hERG. Post-compound currents expressed as a percentage of pre-compound currents plotted against the concentration for each compound.

### Experimental Evidence of ES-3000's Efficacy in Human Leukemias

### Experiment I: Effects of Tetrandrine of β-catenin in Human Leukemia (K562) Cells

TET's ability in an in vitro assay to reduce β-catenin expression was tested in human erythroleukemic (K562) cells. The first portion of the study consisted of cell exposure to Tetrandrine and preparation of cell lysates. Testing was conducted by Applied Immunology, Menlo Park, CA.

Briefly, the method for the first portion of the study was as follows: K562 cells were grown and maintained in medium (RPMI, 10% FBS, Pen Strep and glutamate). Cells were then exposed to TET, 0, 0.1, 1, 5, 10, 20 & 40 μM for 24 hours at 37° C. Following drug exposure, cell viability was checked using trypan blue, followed by cell lysis. Supernatants were collected and test for protein concentration using a Pierce BCA kit. The protein assay tubes were read on a Spectramax at 562 nm and Softmax© software was used for protein concentration analysis. Using 8×10⁶ K562 cells per drug concentration, the protein concentration in the cell lysate was determined to be about 1 mg/ml by BCA protein assay as seen in Table 1.

TABLE 1
Effect of Tetrandrine on protein concentration in K562 Cells

| | |
|---|---|
| Treatment | Protein Recovered |

| | | |
|---|---|---|
| 40 | μM | 0.9 mg |
| 20 | μM | 1.0 mg |
| 10 | μM | 1.1 mg |
| 5 | μM | 1.0 mg |
| 1 | μM | 1.1 mg |
| 0.1 | μM | 1.1 mg |
| 0 | μM | 1.2 mg |

| | |
|---|---|
| Cells alone | 1.2 mg |

### Experiment II Effects of Tetrandrine on Beta-catenin and Beta-actin Levels in Human Myelogenous Leukemia K562 Cell Line

The second portion was to quantitate the levels of β-catenin protein by western blot. TET's ability in an in vitro assay to reduce β-catenin expression was tested in human erythroleukemic (K562) cells. Testing was conducted by Alamo Laboratories, San Antonia, TX.

Briefly, the method to quantitate β-catenin protein by western blot was as follows: Extracts of K562 cells (untreated and treated with various concentrations of tetrandrine) were concentrated to 0.4 ml using Centricon-3 column to yield a protein concentration of approximately 2 mg/ml. Samples were mixed with 5X Laemmli buffer and loaded on 8-16% Tris-Glycine gradient gel, then polyacrylamide gel electrophoresis (PAGE) was run at 85 volts for 100 minutes at 6° C. and proteins were transferred on PVDF membrane at 70 Volts for 85 minutes at 8° C. Membrane was blocked with 5% milk in Tris-buffered Saline containing 0.1% TW-20 (TBST). Membrane was incubated with primary antibody for 18 hours at 4° C. followed by 3 washes, 5 minutes each, with TBST and incubated with secondary antibody in TBST for 2 hours at 25° C. The membrane was washed again with TBST as described above and incubated with ECL-2 substrate for 5 minutes and scanned on Kodak Image Station. After scan, blot was stripped of primary and secondary antibodies and re-probed with anti-beta Actin antibody as described above. Protein bands in WB images were quantitated using Molecular Imaging software pack MIS-4.0 software and tabulated in Excel spreadsheet.

Results indicated that Tetrandrine lowers the β-catenin level in K562 cells in a dose dependent manner as seen in Table 2. It is also important to note the β-catenin protein exists as a doublet in all K562 cell extracts, including untreated cells. The doublet could arise from proteolysis, posttranslational modification or mRNA splicing. A clear β-catenin band was evident in extracts of K562 cells treated with 0 to 10 uM tetrandrine (FIG. 3). However, a convincing β-catenin band was absent in K562 cells treated with 20 and 40 uM tetrandrine. Also, a clear beta-Actin band was evident in all extracts of K562 cells treated with 0 to 40 uM tetrandrine (FIG. 4). Quantitation of the protein-bands in FIGS. 3 and 4, revealed that beta-catenin level was significantly less in extracts of K562 cells treated with >10 uM tetrandrine (FIG. 5); whereas the beta-Actin level in extracts of K562 cells treated with up to 40 uM tetrandrine was not affected significantly by drug treatment (FIG. 6). We conclude that TET reduces β-catenin expression in K562 Cells.

The effect of TET treatment on K562 percent cell viability is depicted in Table 2 and is graphically illustrated in FIG. 2.

TABLE 2
Effect of Tetrandrine on K562 cell viability

| | |
|---|---|
| Treatment | % Viability |

| | | |
|---|---|---|
| 40 | μM | 56.4 |
| 20 | μM | 76.5 |
| 10 | μM | 79.7 |
| 5 | μM | 81.8 |
| 1 | μM | 79.2 |
| 0.1 | μM | 82.4 |
| 0 | μM | 81.1 |

| | |
|---|---|
| Cells alone | 86.7 |

### Experiment III: Effects of Tetrandrine in Leukemia Patient Derived Cells (In Vitro)

Study Title: In Vitro Evaluation of one Test Compound on Human Chronic Myeloid Leukemia Using Colony Forming Cell Assays
Study Report: END 01
Testing Entity: Reach Bio, Seattle, WA

An in vitro investigation was performed to determine whether TET has efficacy on human chronic myeloid leukemia (CML) stem-like cells. A colony-forming cell (CFC) assay was performed on bone marrow cells from a de-novo CML patient.

Human bone marrow light-density cells derived from CML patient bone marrow (Conversant Bio, Alabama), were stored in the gaseous phase of liquid nitrogen until required. On the day of the experiment, the cells were thawed rapidly, the contents of the vial were diluted in 10 mL of Iscove's Modified Dulbecco's Medium containing 10% fetal bovine serum (IMDM+10% FBS) and washed by centrifugation (approximately 1200 r.p.m. for 10 minutes, room temperature). The supernatant was discarded, and the cell pellets were resuspended in a known volume of IMDM+10% FBS. A cell count (3% glacial acetic acid) and viability assessment (trypan blue exclusion test) were treated with Tetrandrine (10 μM) for 14 days. After treatment, primary and secondary colonies were grown and analyzed by qPCR to determine BCR-ABL or ABL only cells.

Human erythroid (CFU-E and BFU-E) and myeloid (CFU-GM) colony enumeration were performed by trained personnel. In addition, the distribution of colony types as well as general colony and cellular morphology were analyzed. The variance in colony number detected in replicate cultures was representative of the historical coefficient of variation for colony enumeration using these types of assays. The number and distribution of colonies detected in the solvent control (0.2% DMSO) was not statistically different from the standard control (containing neither Tetrandrine or Imatinib compounds nor DMSO). For statistical analysis, colony numbers in compound-treated cultures were compared to the solvent control cultures. Potential effects of Tetrandrine and CML progenitors are presented in Table 3.

TABLE 3
Effect of ES-3000 (Tetrandrine) and Imatinib
on CML in a Methylcellulose-based Medium

| | | | Total | | CFU- | |
|---|---|---|---|---|---|---|
| | CFU-E | BFU-E | Erythroid | CFU-GM | GEMM | Total CFC |
| Standard | 10 ± 3 | 23 ± 2 | 33 ± 4 | 60 ± 4 | ND | 93 ± 0 |
| Solvent Control | 10 ± 1 | 23 ± 4 | 33 ± 4 | 56 ± 5 | ND | 89 ± 7 |
| ES-3000 30 μM | ND**** | ND** | ND** | ND** | ND | ND*** |
| ES-3000 10 μM | ND**** | ND** | ND** | 26 ± 2* | ND | 26 ± 2*** |
| ES-3000 3 μM | 9 ± 2 | 14 ± 3 | 23 ± 2 | 52 ± 5 | ND | 75 ± 6 |
| ES-3000 1 μM | 10 ± 2 | 22 ± 4 | 33 ± 3 | 54 ± 2 | ND | 86 ± 4 |
| ES-3000 0.1 μM | 11 ± 1 | 22 ± 4 | 33 ± 3 | 55 ± 5 | ND | 88 ± 8 |
| Imatinib 30 μM | ND**** | ND** | ND** | 5 ± 1** | ND | 5 ± 1*** |
| Imatinib 10 μM | ND**** | ND** | ND** | 12 ± 2** | ND | 12 ± 2*** |
| Imatinib 3 μM | ND**** | ND** | ND** | 27 ± 6 | ND | 27 ± 6** |
| Imatinib 1 μM | ND**** | ND** | ND** | 39 ± 6 | ND | 39 ± 6** |
| Imatinib 0.1 μM | 9 ± 1 | 18 ± 4 | 28 ± 5 | 57 ± 3 | ND | 85 ± 7 |

*p < 0.01;
**p < 0.001;
***p < 0.0001;
****p < 0.00001;
ND none detected

Of interest, the media formulation used for the culture of the progenitors, did not contain erythropoietin so the erythroid colonies which were detected in these cultures represent Epo independent clonal growth. ES-3000 had significant effect on CFU-GM colonies at 30 and 10 μM, while also having the same effect towards the erythroid lineage. The experimental positive control, Imatinib, also had a significant effect on CFU-GM progenitors at 30 and 10 μM but was more toxic to the erythroid lineage (30 to 1 μM) when compared to ES-3000. IC₅₀ values were determined and based on these, Imatinib was more toxic than ES-3000, Table 4.

TABLE 4
Effects of ES-3000 (Tetrandrine) and Imatinib
on Human Progenitors CML marrow

| | Erythroid IC50 ± 95% | Myeloid IC50 ± 95% |
|---|---|---|
| Compound | CI (μM) | CI (μM) |
| ES-3000 | 3.12 ± 0.01 | 9.56 ± 1.25 |
| Imatinib | 0.12 ± 0 | 2.66 ± 0.86 |

### Experiment IV: Replating Assay

This CML sample supported secondary clonal growth as determined through the replating assay (Table 3). In the solvent control cultures, 66.7% wells supported secondary growth with one or more colony and an average of three CFC per well. In the cultures initiated with colonies derived from the original cultures with Imatinib @ 3 μM, 16.7% of wells supported secondary growth with one or more colony and an average of two CFC per well. In the cultures initiated with colonies derived from the original cultures with ES-3000 @10 μM, 54.2% of wells supported secondary growth with one or more colony and an average of three CFC per well.

### Experiment V: qPCR Assay for bcr-abl

To determine whether a colony was normal (bcr-abl negative and abl positive) or diseased (bcr-abl and abl positive), a TaqMan®-based qPCR test was used. In order to score the colonies positive or negative, the following scoring strategy was performed. All colonies were assayed in duplicate for each target. If a singleton reaction yielded a Ct then the reaction scored a 1. If no Ct was recorded, a score of 0 was given. From here, we used a logic test to score the colonies into four different categories: bcr-abl and abl, abl only, bcr-abl only or both negative. In order to be placed in a category, a colony must score a 1 in both reactions. However, if a colony only scored a 1 for one out of the two reactions, this colony was removed from the analysis. If a colony scored a 1 for each reaction (both bcr-abl and abl) the colony was placed into the ber-abl and abl category, signifying it as a diseased colony. Alternatively, if a colony scored a 1 in only both of the bcr-abl reactions or a 1 in only both the abl reactions, the colony was placed in the bcr-abl only or abl only category, respectively. If the colony scored a 0 for both targets (bcr-abl and abl) then the colony was placed in the both negative category.

In total 48 primary colonies and 31 secondary replated colonies were assessed for bcr-abl and abl using qPCR. Using the scoring logic described above, three colonies were removed from the analysis. Thus, this yielded 20 out of 21 colonies (95.2%) scoring positive for both bcr-abl and abl. Seven colonies treated with Tetrandrine at 10 μM were removed from the analysis leaving 11 out of 17 colonies ber-abl and abl positive (64.7%), One out of 17 was ab/positive only (5.8%), while one out of 17 was bcr-abl positive only (5.8) and 4 out of 17 colonies were negative for both targets (23.5%). Most of the secondary colonies treated in solvent control conditions score positive for both bcr-abl and abl (10 out of 14, 71.4%) While one out of 14 (7.1%) was positive for abl only and one out of 14 (7.1%) was positive of bcr-abl only. Two out of 14 colonies scored negative for both targets. Thirteen secondary colonies treated with Tetrandrine at 10 μM were assessed. However, most colonies scored negative for both bcr-abl and abl (9 out of 10, 90%) suggesting the colony's transcript level was out of the level of detection of the assay. Only 1 out of 10 colonies scored positive for abl only (10%). No colonies scored positive for bcr-abl and abl. Further, a few secondary colonies that were treated with Imatinib at 3 μM were assessed for bcr-abl and abl. Only one out of four colonies was positive for both targets, while the remaining three colonies were negative for both targets. The results are provided in Tables 5 and 6. Table 5 indicates that in the solvent control cultures, 66.7% wells supported secondary growth with one or more colony and an average of three CFC per well. In the cultures initiated with colonies derived from the original cultures with Imatinib @ 3 μM, 16.7% of wells supported secondary growth with one or more colony and an average of two CFC per well. In the cultures initiated with colonies derived from the original cultures with ES-3000 @10 μM, 54.2% of wells supported secondary growth with one or more colony and an average of three CFC per well.

TABLE 5
Repeating Colony Count

| Solvent Control | | | | Imatinib @, 3 μM | | | | ES-3000 @ 10 μM | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | | | |
|---|---|---|---|---|---|---|---|---|---|---|---|---|
| A | 3 | 4 | 3 | 3 | 0 | 0 | 0 | 1 | 0 | 0 | 5 | 0 |
| B | 1 | 2 | 1 | 1 | 0 | 0 | 2 | 3 | 0 | 0 | 0 | 2 |
| C | 4 | 0 | 7 | 0 | 0 | 0 | 0 | 0 | 1 | 2 | 5 | 1 |
| D | 4 | 0 | 0 | 1 | 0 | 0 | 0 | 0 | 1 | 0 | 0 | 1 |
| E | 0 | 0 | 0 | 4 | 1 | 0 | 0 | 0 | 1 | 6 | 7 | 2 |
| F | 5 | 1 | 0 | 3 | 0 | 0 | 0 | 0 | 0 | 0 | 0 | 2 |

TABLE 6
bcr-abl Colony Assessment Using qPCR

| | | bcr-abl | | | | | Total |
|---|---|---|---|---|---|---|---|
| | | and abl | | bcr-abl | both | | Colonies |
| | | colonies | abl only | only | negative | Omit | Assessed |
| Primary | Solvent Control | 20 | 0 | 0 | 1 | 3 | 24 |
| | ES - 3000 @ 10 μM | 11 | 1 | 1 | 4 | 7 | 24 |
| Secondary | Solvent Control | 10 | 1 | 1 | 2 | 0 | 14 |
| | ES - 3000 @, 10 μM | 0 | 1 | 0 | 9 | 3 | 13 |
| | Imatinib @ 3 μM | 1 | 0 | 0 | 3 | 0 | 4 |

In conclusion, a semi-solid culture system which supports the proliferation of chronic myeloid progenitors, test compound ES-3000 was assessed and compared with Imatinib on primary and secondary clonal growth. This patient sample supported Epo independent growth (i.e. erythroid colonies were detected in the cultures though Epo was not added to the medium). ES-3000 and Imatinib both inhibited erythroid and myeloid colonies, though the IC50 values for Imatinib were lower than those for ES-3000. Secondary cultures were assessed by plucking individual colonies from the solvent control cultures as well as those from cultures to which Imatinib at 3 μM and ES-3000 at 10 μM were added (representing their respective IC50 values and replating them in fresh culture medium. In the solvent control cultures, 66.7% of colonies supported secondary growth as compared to 16.7% of the Imatinib treated cultures and 54.2% of the ES-3000 treated cultures. Table 6 indicates results of a qPCR assay used to detect bcr-abl transcripts in plucked colonies. 95 percent of colonies from primary plating solvent control cultures were bcr-abl positive compared to 65 percent of colonies treated with ES-3000. Upon analyzing colonies from the secondary replating, most colonies treated with ES-3000 were below the limit of detection while a majority solvent control colonies remained bcr-abl positive.

### Experiment VI: Effect of Tetrandrine in a Leukemia Mouse Model

Study Title: Inhibition of CML Stem Cells with an Alkaloid that Reduces β-catenin
Study Report: In Vivo Report UMass
Testing Entity: University of Massachusetts Medical School (Dr. Li's Lab), Worchester, MA

In an in vivo model, the effectiveness of Tetrandrine was tested on CML stem cells. The objective of the study was to determine the survival of leukemic cells and leukemic stem cells in response to the treatment with Tetrandrine. After priming donor C57BL/6 (B6) mice with intravenous injections of 5-fluorouracil for four days, bone marrow cells were harvested from femurs and tibia, then transfected twice with retrovirus containing MSCV-BCR-ABL-IRES-GFP. Recipient mice were lethally irradiated by two doses of 550 cGy before bone marrow transplantation by intravenous injection with 5×10⁵ cells/mouse. Blood from recipient mice was tested for disease induction one week after transduction by FACS analysis for GFP (FIG. 7A). All mice tested positive. Treatments started on day 8 after bone marrow transplantation (FIG. 7B). Mice were randomized into four groups and treated orally with vehicle [3×/day, 2×], Imatinib [100 mg/kg, 2×/day], Tetrandrine [150 mg/kg, 1×/day] or Imatinib+Tetrandrine [3×/day: 2× with Imatinib, 1× with Tetrandrine].

The results of this study demonstrated that Tetrandrine given orally once a day is superior to Imatinib given twice a day in inhibiting the development of both circulating leukemic cells and leukemic stem cells while the combination of Tetrandrine with Imatinib further improves efficacy of Tetrandrine.

### Experiment VII: Effect of Tetrandrine on CamKIIγ Activity

Study Title: ES-3000 inhibition of CAMKII gamma
Study Report: CamKII Activity Report ES-3000
Testing Entity: Institute for Rare and Neglected Diseases Drug Discovery , Mountain View, CA

An enzymatic study was conducted to evaluate the inhibition of CAMKII gamma by ES-3000 (Tetrandrine). CAMKII gamma kinase activity was measured using ATP GLO purchased from Promega following the Promega protocol. CAMKII enzyme system purchased from Promega (V3531). ADP Glo was used for detection. Assay optimized for linearity of protein and time. Conditions for inhibitor screening 25 ng CAMKII, 20 min. incubations, inhibitor or DMSO (5%) at RT in 20 uL total volume. Inhibitors, ATP (10 or 50 uM), substrates and buffer added to tubes and reactions started with enzyme. Staurosporin was used as positive control. Reactions terminated at 20 min. by adding ADP Glo reaction following manufactures instructions. All reactions were done in duplicate. Calculations of percent (%) inhibition determined by dividing the mean activity at each condition by the mean of the DMSO control times 100.

Results of individual experiments with 10 μM or 50 μM ATP are presented in Table 7 and Table 8, respectively.

TABLE 7
CamKIIγ Activity Inhibition by Tetrandrine with 10 uM ATP

| | | | | | % |
|---|---|---|---|---|---|
| Conditions | units | units | mean | % DMSO | inhibition |
| DMSO control | 4.56 | 4.15 | 4.355 | 100% | 0.1 |
| berbamine 250 uM | 2.38 | 2.3 | 2.34 | 54% | 46.3 |
| berbamine 100 uM | 2.29 | 2.32 | 2.305 | 53% | 47.1 |
| berbamine 30 uM | 2.55 | 2.967 | 2.7585 | 63% | 36.7 |
| ES-3000 250 uM | 0.34 | 0.48 | 0.41 | 9% | 90.6 |
| ES-3000 100 uM | 2.5 | 3.24 | 2.87 | 66% | 34.2 |
| ES-3000 30 uM | 2.75 | 3.43 | 3.09 | 71% | 29.1 |
| Staurosporin 10 nM | 0.93 | 0.9 | 0.915 | 21% | 79.0 |
| Staurosporin 100 nM | 0.25 | 0.15 | 0.2 | 5% | 95.4 |
| DMSO control | 4.16 | | 4.16 | 95% | 4.6 |
| ADP std - 10 uM | 0.52 | 0.44 | 0.48 | | |
| ATP std - 10 uM | 20.2 | 22.8 | 21.5 | | |

TABLE 8
CamKIIγ Activity Inhibition by Tetrandrine with 50 uM ATP

| | | | | % | % |
|---|---|---|---|---|---|
| Condition | units | units | mean | DMSO | inhibition |
| ES-3000 100 uM | 8.81 | 7.1 | 7.955 | 64% | 36 |
| ES-3000 30 uM | 10.4 | 10.7 | 10.55 | 85% | 15 |
| Staurosporin 10 nM | 9.93 | 4.64 | 7.285 | 59% | 41 |
| Staurosporin 100 nM | 0.67 | 0.6 | 0.635 | 5% | 95 |
| DMSO control | 11.5 | 13.3 | 12.4 | 1 | |
| ATP std - 10 uM | 0.34 | 0.37 | 0.355 | | |
| ADP std - 10 uM | 9.09 | 7.15 | 8.12 | | |

The results from the study showed a good signal to noise. Staurosporin was effective as a positive control and ES-3000 inhibited CAMKII gamma kinase activity in a dose dependent manner.

Table 9 represents inhibition of activity CamKIIγ in the presence of various concentration of ES-3000 (Tetrandrine), staurosporin and berbamine indication dose-dependent inhibition.

TABLE 9
CamKIIγ Activity Inhibition by Tetrandrine

| | Percent inhibition | Percent inhibition |
|---|---|---|
| | 10 uM ATP | 50 uM ATP |
| ES-3000 250 uM | 91 | |
| ES-3000 100 uM | 34 | 36 |
| ES-3000 30 uM | 29 | 15 |
| staurosporin 100 nM | 95 | 95 |
| staurosporin 10 nM | 79 | 41 |
| berbamine 250 uM | 46 | |
| berbamine 100 uM | 47 | |
| berbamine 30 uM | 37 | |

### Experiment VIII Effects of TET on hERG

Study Title: hERG Inhibition Potential by the Test Article ES-3000
Study Report: CYP1328_R1
Testing Entity: Cyprotex US, LLC, Watertown, MA

The purpose of this study was to evaluate the potential of a test article to inhibit hERG. The experiments are performed on an IonWorks™ HT instrument (Molecular Devices Corporation), which automatically performs electrophysiology measurements in 48 single cells simultaneously in aspecialized 384-well plate (PatchPlate). All cell suspensions, buffers and test compound solutions are at room temperature during the experiment. The cells used are Chinese hamster ovary (CHO) cells stably transfected with hERG (cell-line obtained from Cytomyx, UK). A single-cell suspension is prepared in extracellular solution (Dulbecco's phosphate buffered saline with calcium and magnesium pH 7-7.2) and aliquots added automatically to each well of a PatchPlate™. The cells are then positioned over a small hole at the bottom of each well by applying a vacuum beneath the plate to form an electrical seal. The vacuum is applied through a single compartment common to all wells which is filled with intracellular solution (buffered to pH 7.2 with HEPES). The resistance of each seal is measured via a common ground-electrode in the intracellular compartment and individual electrodes placed into each of the upper wells. Electrical access to the cell is then achieved by circulating a perforating agent, amphotericin, underneath the PatchPlate and then measuring the pre-compound hERG current. An electrode is positioned in the extracellular compartment and a holding potential of −80 mV applied for 15 sec. The hERG channels are then activated by applying a depolarizing step to +40 mV for 5 sec and then clamped at −50 mV for 4 sec to elicit the hERG tail current, before returning to −80 mV for 0.3 sec. Test compound is then added automatically to the upper wells of the PatchPlate™ from a 96-well microtitre plate containing a range of concentrations of each compound. Solutions are prepared by diluting DMSO solutions of the test compound into extracellular (final DMSO concentration 0.25%). The test compound is left in contact with the cells for 300 sec before recording currents using the same voltage-step protocol as in the pre-compound scan. Quinidine, an established hERG inhibitor, is included as a positive control and buffer containing 0.25% DMSO is included as a negative control. The results for all compounds on the plate are rejected and the experiment repeated if the IC50 value for quinidine or the negative control results are outside quality-control limits. Each concentration is tested in 4 replicate wells on the PatchPlate™. However, only cells with a seal resistance greater than 50 mOhm and a pre-compound current of at least 0.1 nA are used to evaluate hERG blockade. Post-compound currents are then expressed as a percentage of pre-compound currents and plotted against concentration for each compound (FIG. 8). Testing was conducted by Cyprotex US, LLC, Watertown, MA. Experimental conditions (Table 10) and results (Table 11) are presented below.

TABLE 10
hERG Inhibition Experimental Conditions

| | | Reference | |
|---|---|---|---|
| Test Article | Test Conc. | Compound | Analytical Method |
| ES-3000 | .004-12.5 μM | Quinidine | electrophysiology |

TABLE 11
Results of hERG Inhibition by ES-3000

| | Test Conc. | | | |
|---|---|---|---|---|
| Test Article | (μM) | IC₅₀ (μM) | SE IC₅₀ | Comment |
| Quinidine | 0.01-10 | 1.66 | 0.353 | Positive Control |
| ES-3000 | 0.004-12.5 | 5.15 | 0.699 | |

### Discussion and Conclusions from Experimental Studies

This disclosed invention relates to novel compositions and methods to improve the utility of chemical agents, biological therapies including bisbenzylisoquinolines such as 6,6′,7,12-tetramethoxy-2,2′-dimethyl-berbaman (tetrandrine) and derivatives with suboptimal performance for patients with cancer, infection and immunological diseases. The disclosed invention describes the novel development of improved pharmaceutical ingredients, dosage forms, excipients, solvents, diluents, drug delivery systems, preservatives, more accurate drug administrations, improved dose determination and schedules, toxicity monitoring and amelioration, techniques or agents to circumvent or reduce toxicity, techniques and tools to identify/predict those patients who might have a better outcome with a therapeutic agent by the use of phenotype or genotype determination through the use of diagnostic kits or pharmacokinetic or metabolism monitoring approaches , the use of drug delivery systems, novel prodrugs, polymer conjugates, novel routes of administration, other agents to potentiate the activity of the compounds or inhibit the repair of suboptimal cellular effects or sub-lethal damage or to “push” the cell into more destructive cellular phases such as apoptosis. In some cases, the inventive examples include the use of these sub-optimal therapeutics in conjunction with radiations or other conventional chemotherapeutic agents or biotherapeutic agents such as antibodies, vaccines, cytokines, lymphokines, gene and antisense therapies, etc.

As used herein, “suboptimal” refers to “less that desired” and the terms “suboptimal therapy” and “suboptimal therapeutics” include agents where Phase I toxicity precluded further human clinical evaluation, agents from Phase II trials where limited or no significant tumor responses were identified, and agents from Phase III clinical trials, whose outcome was either medically or statistically not significant to warrant submission or approval by regulatory agencies for commercialization or commercialized agents whose response rates as a monotherapy are less than 25% or whose side-effects are severe enough to limit wider utility. In addition, it includes, allergic reaction, adverse or fatal side effects, organ damage, due to suboptimal therapeutics. Agents with suboptimal activity include but are not limited to the following: bisbenzylisoquinolines including 6,6′,7,12-tetramethoxy-2,2′-dimethyl-berbaman (tetrandrine) and derivatives, Avastin® (bevacizumab), Rituxan® (rituximab), Neavar® (sorafenib), dasatinib, imatinib, nilotinib, Provenge® (sipuleucel-T), Tarceva® (erlotinib), and Iressa® (gefitinib). More specifically, the inventive methods and compositions also focus on improvements for bisbenzylisoquinolines including 6,6′,7,12-tetramethoxy-2,2′-dimethyl-berbaman (tetrandrine) and derivatives.

Tetrandrine or TET has been shown to have multiple pharmacological activities relating to anticancer functions. Those include inhibition of Wnt/β-catenin, reduction of expression of MDR1 and inhibition of P-gp function, activation of apoptosis, and suppression of proliferation via various activation and survival signaling pathways. We have further investigated some of the pharmacologic properties most relevant to the use of TET for leukemia. Results from our studies demonstrated that TET reduced β-catenin in human (K562) leukemia cells, reduced bcr-able positive primary colonies and eliminated bcr-able positive secondary colonies in a colony forming cell assay. In a murine model of leukemia, TET reduced the development of leukemic cells in the peripheral blood and leukemic stem cells in the bone marrow of mice. In an vitro enzymatic assay, TET inhibited the activation of CaMKIIγ in a dose dependent manner.

In vitro evaluation of TET's ability to inhibit hERG, revealed an IC₅₀ of 5.2 μM, which is 13-fold higher safety for cardiac conduction-related toxicity utilizing Cₘₐₓ from previous human clinical experience (Oldham 1998, Oldham 2000). Though, QT changes may be observed at concentrations lower than IC₅₀, a reasonable interpretation is that we might expect to see QT prolongation in subjects at plasma levels above 249 ng/ml, as Tetrandrine is highly protein bound.

Additionally, the Wnt/β-catenin pathway has been identified as a new target for AML (Wang 2010) and ES-3000 has shown to reduce β-catenin. More recent studies have shown that TET is able to bind competitively to calmodulin (CaM) and may be a novel CaM antagonist (Ma 2013). Calmodulin protein dependent kinase (CaMKIIγ) is overexpressed in leukemic stem cells and blast cells, and regulates the Wnt/β catenin and STAT3 pathways (Si 2008). An evaluation of β-catenin and CaMKIIγ levels in patients to determine a correlation between response and expression levels may provide valuable information in selecting patients who would benefit most from ES-3000 treatment.

The multiplicity of tetrandrine's mechanisms and safety profile provides an opportunity for its development as a therapeutic for relapsed or refractory myeloid leukemias and related hematological diseases.

### LIST OF ABBREVIATIONS

| Abbreviation | Description |
|---|---|
| aCML | Atypical chronic myeloid leukemia |
| AE | Adverse event |
| AML | Acute myeloid leukemia |
| AML | Acute myeloid leukemia |
| ATP | Adenosine triphosphate |
| B-ALL | B-cell acute lymphoblastic leukemia |
| BBL | Bisbenzylisoquinolines |
| CaMKIIγ | Calmodulin protein dependent kinase gamma |
| CcyR | Complete cytogenetic response |
| CML | Chronic myeloid leukemia |
| CMML | Chronic myelomonocytic leukemia |
| CMR | complete molecular response |
| CR | Complete response |
| EMA | European medicine agency |
| ES-3000 | Tetrandrine, d-tetrandrine, NSC 77037 |
| FDA | Food and Drug Administration |
| FDA | Food and Drug Administration |
| GCSF | granulocyte-colony stimulating factor |
| IM | Imatinib mesylate |
| IS | International Scale |
| IV | Intravenous (IV) |
| JMML | Juvenile myelomonocytic leukemia |
| Kg | Kilogram |
| MCR | Major cytogenetic response |
| MDS | Myelodysplastic syndrome |
| MDS-U | MDS, unclassifiable |
| mg | Milligrams |
| mg/m2 | Milligrams per square meter |
| MMR | Major molecular response |
| MPN | Myeloproliferative neoplasm |
| MRD | Minimum residual disease |
| NCI | National cancer institute |
| PO | Orally |
| PR | Partial remission |
| PV | Polycythemia vera |
| qRT-PCR | quantitative real-time PCR |
| R/R | Relapsed or refractory |
| RAEB-1 | Refractory anemia with excess blasts-1 |
| RAEB-2 | Refractory anemia with excess blasts-2 |
| RARS | Refractory anemia with ring sideroblasts |
| RBC | Red blood cell |
| RCMD | Refractory cytopenia with multilineage dysplasia |
| RCUD | Refractory cytopenia with unilineage dysplasia |
| RP2D | Recommended Phase II dose |
| RR | Response rate |
| SAE | Serious adverse event |
| TET | Tetrandrine (6,6′,7,12-tetramethoxy-2,2′-dimethylberbaman) |
| TID | Three times per day |
| TKI | Tyrosine kinase inhibitor |
| TNBC | Triple negative breast cancer |
| WBC | White blood cell |

### REFERENCES

Arcangeli A, Becchetti A, New trends in cancer therapy: targeting ion channels and transporters. Pharmaceuticals, 2010. 3:1202-1224.
Arcangeli A, Pillozzi S, Becchetti A, Targeting ion channels in leukemias: a new challenge for treatment. Curr Med Chem, 2012. 19(5):683-96.
Döhner H, Weisdorf DJ, Bloomfield CD, Acute Myeloid Leukemia. The New England Journal of Medicine, 2015. 373(12):1136-1152.
Fanelli M, Hattinger CM, Vella S, Tayanti E, Michelacci F, Gudeman B, Barnett D, Picci P, Serra M, Targeting ABCB1 and ABCC1 with their specific inhibitor CBT-1 can overcome drug resistance in osteosarcoma. 2016, Curr Canc Drug Targets16:261-274.
Gralla EJ, Coleman GL, Jonas AM, Toxicology studies with d-Tetrandrine (NSC-77037), a plant alkaloid with vascular and lymphotoxic effects in dogs and monkeys. Cancer Chemotherapy Report Part 3, 1974. 5:79-84.
Griffiths EA, Gore SD, Hooker CM, et al., Acute myeloid leukemia is characterized by Wnt pathway inhibitor promoter hypermethylation. Leuk & Lymph, 2010. 51(9): 1711-1719.
He BC, Gao J-L, Zhang B-Q, et al. Tetrandrine inhibits Wnt/β-catenin signalling and suppresses tumor growth of human colorectal cancer. Mol Pharmacol, 2011. 79(2):211-9.
Huang Y-T, Hong C-Y, Tetrandrine. Cardiovascular Drug Reviews, 1998. 16(1):1-15.
Huber SM, Oncochannels. Cell Calcium, 2013. 53(4):241-55.
Jin J, Wang FP, Wei H and Liu G. Reversal of multidrug resistance of cancer through inhibition of P-glycoprotein by 5-bromoTetrandrine. Cancer Chemotherapy and Pharmacology. 2005. 55:179-188.
Ju A, Kang Y, et al. Pharmacokinetics, tissue distribution and excretion study of tetrandrine in rats. Journal of Chinese Pharmaceutical Sciences. 2015;24(8).
Kelly RJ, Robey RW, Chen CC, Draper D, Luchenko V, Barnett D, Oldham RK, Caluag Z, Frye AR, Steinberg SM, Fojo T, Bates SE, A pharmacodynamic study of the P-glycoprotein antagonist CBT-1 in combination with paclitaxel in solid tumors. 2012, The Oncologist. 17:512.
Lang F, Stoumaras C, Ion channels in cancer: future perspectives and clinical potential. Phil Trans R Soc B, 2014. 369: 20130108.
Liu W, Zhang J, Ying C, et al. Tetrandrine combined with gemcitabine and cisplatin for patients with advanced non-small cell lung cancer improve efficacy. Int J Biomed Sci, 2012. 8(1):28-35.
Ma L, Wang Z, Liu S, et al. Screening calmodulin-binding ligands using intensity-fading matrix-assisted laser desorption/ionization mass spectrometry. Rapid Commun Mass Spectrom, 2013. 27: p. 1527-1534.
National Cancer Institute, Annual Progress Report for IND #089613, 1976.
Ng LT, Chiang L-C, Lin Y-T, et al. Antiproliferative and apoptotic effects of Tetrandrine on different human hepatoma cell lines. Am J Chin Med, 2006. 34(1):125-35.
Oldham RK, Reid WK, Preisler HD, Barnett D. A phase I and pharmacokinetic study of CBT-1 as a multidrug resistance modulator in the treatment of patients with advanced cancer. Cancer Biother Radiopharm. 1998; 13:71-80.
Oldham RK, Reid WK, Barnett D, Phase I study of CBT-1 and Taxol in patients with Taxol resistant cancers. 2000, Cancer Biother Radiopharm. 15(2):153-159.
Si J, Collins SJ, Activated Ca2+/Calmodulin-dependent protein kinase IIγ is a critical regulator of myeloid leukemia cell proliferation. Cancer Res, 2008. 68(10):3733-3742.
Simon M, Grandage VL, Linch DC and Khwaja A. Constitutive activation of the Wnt/β-catenin signalling pathway in acute myeloid leukaemia. Oncogene 2005. 24:2410-2420.
Susa M, Choy E, Yang C, Schwab J, Mankin H, Hornicek F, Duan Z. 2010. Multidrug resistance reversal agent, NSC77037, identified with a cell-based screening assay. J Biomol Screen. 15, 287-296.
Wang G, Lemos JR, Tetrandrine: a new ligand to block voltage-dependent Ca2+ and Ca2+− activated K+ channels. Life Sci, 1995. 56(5):295-306.
Wang Y, Krivtsov A, Sinha A, The wnt/β-catenin pathway is required for the development of leukemia stem cells in AML. Science, 2010. March 26; 327(5973):1650-1653.
Wu J-M, Chen Y, Chen J-C, Lin T-Y, Tseng S-H, Tetrandrine induces apoptosis and growth suppression of colon cancer cells in mice. 2010, Cancer Letters. 287:187-195.
Wu K, Zhou M, Wu Q-X, Yuan S-X, Wang D-X, Jin J-L, Huang J, Yang J-Q, Sun W-J, Wan L-H, He B-C, The role of IGFBP-5 in mediating the antiproliferation effects of Tetrandrine in human colon cancer cells. 2015, Int. J Oncology. 46:1203-1213.
Xu W-L, Shen H-L, Ao Z-F, Combination of Tetrandrine as a potential-reversing agent with daunorubicin, etoposide and cytarabine for the treatment of refractory and relapsed acute myelogenous leukemia. Leuk Res, 2006. 30:407-413.
Xu XH, Gan YC, Xu GB, Chen T, Zhou H, Tang JF, Gu Y, Xu F, Xie, YY, Zhao XY, Xu RZ, Tetrandrine citrate eliminates imatinib-resistant chronic myeloid leukemia cells in vitro and in vivo by inhibiting Bcr-Abl/β-catenin axis. J Zhejiang Univ-Sci B (Biomed & Biotechnol), 2012 13(11):867-874.
Zheijiang Jinhua Conba Bio-Pharma, Co. Ltd., (Jinhua, China), Tetrandrine Tablet Package Insert 2014.
Zhu X, Sui M, Fan W, In vitro and in vivo characterizations of Tetrandrine on the reversal of p-glycoprotein-mediated drug resistance to paclitaxel. 2005, Anticancer Research. 25:1953-1962

What is claimed:

1. Methods for treating preneoplastic and neoplastic diseases and solid tumors in a subject in need thereof comprising administering to the subject a compound of Formula I (tetrandrine) comprising the following structural formula: 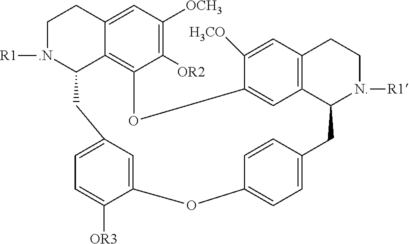
a. where R₁ and R₁′ are the same or different short chained carbon-based ligands including without limitation, CH₃, CO₂CH₃ or H; and R₂ is CH₃ or C₂H₅; and R₃ is CH₃ or hydrogen, has the “S” isomeric configuration at the C-1′ chiral carbon location, and
b. wherein the disease is selected from the group consisting of myelodysplastic syndrome (MDS), myeloproliferative neoplasms (MPN), leukemia and solid tumors and
c. wherein an effective amount comprises 0.01 to 1000 mg/m².

2. The method of claim 1, wherein the disease is selected from the group consisting of myelodysplastic syndrome (MDS) chronic myeloid leukemia (CML), atypical chronic myeloid leukemia (aCML), and acute myeloid leukemia (AML), polycythemia vera (PV).

3. The method of claim 2, wherein the patient is administered tetrandrine
a. before treatment with a tyrosine kinase inhibitor or other chemotherapeutic regimens, or immunologic therapeutics;
b. after treatment with the tyrosine kinase inhibitor, or other chemotherapeutic regimens, or immunologic therapeutics; or
c. contemporaneously with the tyrosine kinase inhibitor or other chemotherapeutic regimens or immunologic therapies;
d. wherein the leukemia is resistant intrinsical or extra-intrinsical and has minimal residual disease after treatment with a tyrosine kinase inhibitor, or other chemotherapeutic regimens, or is refractory to or has resistance to a tyrosine kinase inhibitor or other chemotherapeutic regimen.

4. The method according to claim 1, wherein the disease is affected by at least one of:
a. treatment through the inhibition or killing of cancer stem cells or leukemic stem cells,
b. treatment through the inhibition of molecular pathways including selected from the group consisting of Alox5, Stat3, Wnt/beta-catenin, Msr2, Blk, Myc, Survivn, Cyclin D, Osteopontin, Tenascin C, L1CAM and CaMKII, and
c. treatment through the inhibition of molecular protein targets involved in the survival of cancer and leukemia stem cells selected from the group consisting of lipoxygenases, Mcl-1, cyclin-D1, beta-catenin, Bcl-2, Bcl-xL NF-kappaB, CamKIIγ, pCaMKIIγ and VEGF.

5. The method of claim 3, further comprising administering an additional anti-leukemia agent(s), wherein said additional anti-leukemia agent(s)is selected from existing approved treatments for leukemia, MDS or MPN and wherein said additional anti-leukemia agent is selected from one or more of tyrosine kinase inhibitors, SRC-kinase inhibitors, JAK2 kinase inhibitors, aurora kinase inhibitors, interferon alpha, hydroxyurea, anthracyclines, cytarabine, ara-C, daunorubicine or doxorubicin and combinations of two or more anti-leukemia agents.

6. The method of claim 1, wherein the method of tetrandrine is administered by at least one of:
a. orally,
b. parenterally (infusion IV), and/or
c. subcutaneously.

7. The method of claim 1, wherein the frequency of tetrandrine administration is by at least one of:
a. for hours to days by infusion;
b. biweekly;
c. progressive escalation of dosing based on patient tolerance;
d. selected and intermittent boost dose administrations;
e. dosing by a bolus, single, multiple daily and multiple doses/day, and
f. release of the does that is at least one of immediate, slow, and controlled.

8. The method of claim 1, wherein the bisbenzylisoquinoline is administered daily.

9. The method of claim 1, wherein the bisbenzylisoquinoline is administered for 7-14 days or more.

10. The method of claim 1, wherein the bisbenzylisoquinoline is administered to said patient in an amount of about 0.01 mg/m² to 1000 mg/m².

11. The method according to claim 1, wherein said administration is twice daily for seven days or more.

12. The method according to claim 10 wherein the dose of said the bisbenzylisoquinoline is about 0.01 mg/m² to 1000 mg/m².

13. The method according to claim 1, wherein said the bisbenzylisoquinoline is administered for 7-14 days or more.

14. The method according to claim 11, wherein the dose of said the bisbenzylisoquinoline is about 0.01 mg/m² to 1000 mg/m².

15. A method of administering to a patient in need of myeloablative conditioning prior to hematopoietic stem cell transplantation (HSCT) procedures, said method comprising:
a. Administering a bisbenzylisoquinoline to said patient prior to administration of one or more myeloablative conditioning agents or procedures including:
i. Cyclophosphamide
ii. Busulfan
iii. Radiation
iv. Fludarabine
v. Melphalan,
vi. Clofarabine,
vii. Amsacrine,
viii. Cytarabine
ix. Decitabine
x. Cedazuridine.

16. Methods for treating a subject with cancer stem cell-related preneoplastic and neoplastic diseases such as myelodysplastic syndrome (MDS), myeloproliferative neoplasms (MPN), chronic myeloid leukemia (CML), atypical chronic myeloid leukemia (aCML), acute myeloid leukemia (AML), polycythemia vera (PV) and lymphomas, non-leukemic solid tumors, comprising the step of administering to said subject in recognized need of said treatment an effective amount of bisbenzylisoquinolines such as 6,6′,7,12-tetramethoxy-2,2′-dimethyl-berbaman (tetrandrine) and derivatives, isomers, and analogs, prodrugs, esters, modified forms such as crystalline, salt forms, or a salt of this compound with a pharmaceutically acceptable acid or in combination with other agents.

17. The method of claim 15, wherein tetrandrine or in the form of a derivative, isomer and/or analog, a modified form such as crystalline, salt forms, or a salt of this compound with a pharmaceutically acceptable acid or in combination with a first other agent(s) and is administered along with a pharmaceutically acceptable acid, carrier and/or in a pharmaceutically compatible product or in combination with a second other agent(s).

18. The method of claim 17, wherein said first other agents include Alemtuzumab, Ambochlorin (Chlorambucil), Amboclorin (chlorambucil), Arzerra (Ofatumumab), Bendamustine Hydrochloride, Campath (Alemtuzumab), Chlorambucil, Fludara (Fludarabine Phosphate) Fludarabine Phosphate, Gazyva (Obinutuzumab, Ibrutinib, Imbruvica (Ibrutinib), Leukeran (Chlorambucin), Linfolizin (Chlorambucil), Obinutuzumab, Azacitidine, Decitabine, Inqovi (Cedazuridine and Decitabine), Cedazuridine, Lenalidomide, Doxorubicin HCL, Arsenic Trioxide, Daunorubicin Hydrochloride, Cyclophosphamide, Cytarabine, Cytosar-U (Cytarabine), Cytoxan (Cyclophosphamide), JAK2 inhibitors (e.g., Fedratinib, deucravacitinib, Ritlecitinib), Ketruda (pembrolizumab) Neosar (Cyclophosphamide), Obdivo (nivolumab) Rubidomycin (Daunorubicin Hydrochloride), Tarabine PFS (Cytarabine), Trisenox (Arsenic Trioxide), Vincasar PFS (Vincristine Sulfate), Vincristine Sulfate, Bosulif (Bosutinib), Bosutinib, Busulfan, Busulfex (Busulfan), Clafen (Cyclophosphamide), Dasatinib, Gleevec (Imatinib Mesylate), Iclusig (Ponatinib Hydrochloride), Imatinib Mesylate, Myleran (Busulfan), Nilotinib, Omacetaxine Mepesuccinate, Ponatinib Hydrochloride, Sprycel (Dasatinib), Synribo (Omacetaxine Mepesuccinate), Tarabine PFS (Cytarabine), Tasigna (Nilotinib), Bortezomib (Velcade), Panobinostat, CAR-TCR therapies and related agents.

19. The method and compositions according to claim 1, wherein the compound is as a powder, tablet, capsule, liquid, patch or medical food(s).

20. The method according to claim 1, where the schedule of administration for bisbenzylisoquinolines such as 6,6′,7,12-tetramethoxy-2,2′-dimethyl-berbaman (tetrandrine) and derivatives, isomers and analogs, modified forms such as salt forms, or a salt of this compound with a pharmaceutically acceptable acid or in combination with other agents includes: daily; weekly; weekly for three weeks; biweekly; biweekly for three weeks with a 1-2 week rest period; intermittent boost dose administration; daily for one week for multiple weeks; with or and? Immediate, slow, or controlled release oral administration.

21. A method for treating a subject having chronic phase BCR-ABL-induced myeloid leukemia characterized by oncogene-mediated elevated beta-catenin comprising: administering a therapeutically effective amount of a beta-catenin inhibitor to the subject; and administering a therapeutically effective dose of a BCR-ABL tyrosine kinase inhibitor to the subject, wherein leukemia stem cells are inhibited.

22. The method of claim 21, wherein the beta-catenin inhibitor is a bisbenzylisoquinoline such as 6,6′,7,12-tetramethoxy-2,2′-dimethyl-berbaman (tetrandrine) and derivatives, isomers and analogs, modified forms such as salt forms, or a salt of this compound is administered with a pharmaceutically acceptable acid or in combination with other agents.

23. The method according to claim 21, wherein the BCR-ABL kinase inhibitor is selected from the group consisting of: imatinib, dasatinib, nilotinib, bosutinib, ponatinib, lestaurtinib, tozasertib, danusertib, vatalanib, Nexavar (sorafenib tosylate), Midostaurin, AZD0530 (Saracatinib), NPB-001-05 (imatinib-resistant Bcr-Abl inhibitor), AT9283 (aurora kinase inhibitor), bafetinib, XL228 (multi-targeted protein kinase inhibitor), KW-2449 (multikinase inhibitor), AT-9283 (multi-targeted protein kinase inhibitor), VE-465 (Aurora kinase inhibitor), DCC-2036 (Rebasitinib), and PF-03814735 (Aurora kinase inhibitor).

24. The method of claim 21, further comprising administering a therapeutically effective dose of a beta-catenin inhibitor.

25. The method of claim 21, further comprising administering a therapeutically effective dose of an Hsp90 inhibitor.

26. The method of claim 21, further comprising administering a therapeutically effective dose of an anti-cancer agent selected from the group consisting of: a protein synthesis inhibitor, an anti-metabolite, an alkylating agent, a steroid, interferon alpha 2b, and a combination of any two or more thereof.

27. The method of claim 21, wherein the subject is resistant to anti-cancer effects of a therapeutic agent selected from the group consisting of: imatinib, dasatinib and nilotinib.

28. The method of claim 21, further comprising administering a therapeutically effective dose of a JAK2.

29. The method of claim 28, wherein the JAK2 inhibitor includes but is not limited to Fedratinib, deucravacitinib, Ritlecitinib.

30. The method of claim 21, further comprising administering a therapeutically effective dose of a FLT-3 inhibitors.

31. The method of claim 30, wherein the FLT-3 inhibitors including but is not limited to Sorafenib, Midostaurin, and Lestaurtinib, gilteritinib, quizartinib, crenolanib.

32. The method according to claim 1, wherein R1 and R′ are the same or different short chained carbon-based ligands including without limitation, CH₃, CO₂CH₃ or H; and R₂ is CH₃ or C₂H₅; and R₃ is CH₃ or hydrogen, has the “S” isomeric configuration at the C-1′ chiral carbon location, and wherein the disease is selected from the group consisting of myelodysplastic syndrome (MDS), myeloproliferative neoplasms (MPN), leukemia and solid tumors and wherein an effective amount comprises 0.01 mg/m² to 1000 mg/m².

33. The method according to claim 1, wherein R₁ and R₁′ or R₂ are aliphatic, aromatic, heterocyclic, halide substituted amino, sulfhydryl, carboxylic, ketone, esters, carboxamides esters salts, sugar peptide protein, nucleic acid, antisense antibody substituents.

34. The method according to claim 1, wherein R₁ and R₁′ or R₂ are prodrugs, esters or salt forms.

35. The method according to claim 5, wherein the additional anti-leukemic agents are combined in a single dosage form.

36. The method according to claim 16, wherein the agents are combined in a single dosage form.
